(19) United States
(12) Patent Application Publication
(10) Pub. No.: US 2025/0052751 A1
(43) Pub. Date: Feb. 13, 2025

(54) COMPOSITIONS AND METHODS FOR DIAGNOSING AUTOIMMUNE AQUAPORINOPATHY

(71) Applicant: The Regents of the University of California, Oakland, CA (US)

(72) Inventors: Scott S. Zamvil, San Francisco, CA (US); Sharon A. Sagan, San Francisco, CA (US); Joseph J. Sabatino, San Francisco, CA (US); Carson E. Moseley, San Francisco, CA (US); Collin M. Spencer, San Francisco, CA (US)

(21) Appl. No.: 18/775,367

(22) Filed: Jul. 17, 2024

Related U.S. Application Data
(60) Provisional application No. 63/527,287, filed on Jul. 17, 2023.

(51) Int. Cl. G01N 33/569; G01N 33/564

(52) U.S. Cl. CPC G01N 33/56972; G01N 33/564; G01N 2333/70539; G01N 2800/24; G01N 2800/52

(57) ABSTRACT

Compositions, methods, and kits are provided for diagnosing autoimmune aquaporinopathy. In particular, cell-based assays are provided for detecting autoreactive T cells that bind to certain pathogenic T cell epitopes of aquaporins. The disclosed methods can be used for diagnosing aquaporinopathies, such as neuromyelitis optica spectrum disorder and Sjögren's syndrome.

### CROSS REFERENCE TO RELATED APPLICATIONS

This application claims benefit of U.S. Provisional Patent Application No. 63/527,287, filed Jul. 17, 2023, which application is incorporated herein by reference in its entirety.

### STATEMENT REGARDING FEDERALLY SPONSORED RESEARCH OR DEVELOPMENT

This invention was made with government support under Grant No. RO1 AI131624-01A1 awarded by the National Institutes of Health. The government has certain rights in the invention.

### INCORPORATION BY REFERENCE OF A SEQUENCE LISTING

A Sequence Listing is provided herewith as a Sequence Listing XML file, “UCSF-746” created on Jul. 16, 2024 and having a size of 9,438 bytes. The contents of the Sequence Listing XML file are incorporated by reference herein in their entirety.

### BACKGROUND OF THE INVENTION

Aquaporin-4 (AQP4), a member of the ubiquitous family of water channels (Agre et al., J Physiol 542, 3-16 (2002)), is the principal autoantigen target for pathogenic antibodies in neuromyelitis optica spectrum disorder (NMO/NMOSD), a central nervous system (CNS) autoimmune disease that may cause paralysis and blindness (Zekeridou et al., Neurol Neuroimmunol Neuroinflamm 2, e110 (2015)). AQP4 is expressed abundantly on astrocyte end-foot processes at the blood-brain barrier (Nielsen et al., J Neurosci 17, 171-180 (1997)), the site of CNS damage in NMO, but it is also expressed in kidney, muscle, lung and heart (Agre et al., supra; Magni et al., Proteomics 6, 5637-5649 (2006); Azad et al., Front Genet 12, 654865 (2021)), tissues that are characteristically unaffected in NMO. Evidence suggests T cells may have an important role in NMO pathogenesis (Zekeridou et al., supra; C. F. Lucchinetti et al., Brain Pathol 24, 83-97 (2014)). AQP4-specific antibodies in NMO serum are IgG1, a T cell-dependent IgG subclass (Nurieva et al., Cell Mol Immunol 7, 190-197 (2010)). T cells can be identified in NMO lesions and it has been established that CNS inflammation induced by T cells permits CNS entry of AQP4-specific antibodies (Bennett et al., Ann Neurol 66, 617-629 (2009); Bradl et al., Ann Neurol 66, 630-643 (2009)) (8, 9). NMO susceptibility is also associated with allelic MHC II genes, in particular HLA-DR17 (DRB1*0301) (Brum et al., Mult Scler 16, 21-29 (2010)), and AQP4-specific HLA-DR-restricted CD4⁺ Th17 cells can be identified in the peripheral blood of NMO patients (Varrin-Doyer et al., Ann Neurol 72, 53-64 (2012); Vaknin-Dembinsky et al., Neurology 79, 945-946 (2012)). While these observations indicate AQP4-specific T cells contribute to NMO pathogenesis, they do not address mechanisms responsible for the generation and regulation of those T cells.

Substantial effort has been devoted to development of NMO animal models in order to investigate how AQP4-specific T cells and antibodies participate in cellular and humoral CNS autoimmune responses in vivo. Those efforts have met with limited success (Nelson et al., PLOS One 5, e15050 (2010); Kalluri et al., PLOS One 6, e16083 (2011); Zeka et al., Acta Neuropathol 130, 783-798 (2015); Pohl et al., Acta Neuropathol 122, 21-34 (2011); Jones, et al., Acta Neuropathol Commun 3, 28 (2015); Sagan et al., Proc Natl Acad Sci USA 113, 14781-14786 (2016); Vogel et al., Eur J Immunol 47, 458-469 (2017)). Unlike experimental autoimmune encephalomyelitis (EAE), which is induced either by direct immunization of wild-type (WT) mice with myelin proteins or by adoptive transfer of WT myelin-specific T cells, attempts to create an AQP4-targeted NMO model in WT mice or rats by either direct immunization of AQP4 or its peptides, or by adoptive transfer of WT AQP4-specific T cells were unsuccessful. However, two AQP4 determinants that were predicted to bind MHC II (I-A^{b}) avidly, elicited robust proliferative T cell responses in AQP4-deficient (AQP4^{-/-}) mice, but not in WT mice (Sagan et al. (2016), supra). When used as donor T cells, Th17-polarized AQP4 peptide-specific cells from AQP4^{-/-} mice, but not from WT mice, induced paralysis and CNS inflammation in WT mice lasting several days (FIG. 1) (Sagan et al. (2016), supra; Sagan et al., J Vis Exp 10.3791/56185 (2017)). Thus, mechanisms of tolerance that control T cell reactivity to AQP4 and myelin autoantigens may be distinct.

Thymic negative selection and peripheral regulation are two distinct processes that safeguard against T cell reactivity to tissue specific antigens. The inability to generate pathogenic AQP4-reactive T cells from WT mice suggested that development of AQP4-reactive T cells may be prevented by thymic negative selection.

### SUMMARY OF THE INVENTION

Compositions, methods, and kits are provided for diagnosing autoimmune aquaporinopathy. In particular, cell-based assays are provided for detecting autoreactive T cells that bind to certain pathogenic T cell epitopes of aquaporins. The disclosed methods can be used for diagnosing aquaporinopathies, such as neuromyelitis optica spectrum disorder and Sjögren's syndrome.

In one aspect, a method of detecting an autoreactive T cell associated with an autoimmune aquaporinopathy is provided, the method comprising: a) contacting a population of CD4⁺ T cells with a natural antigen-presenting cell or an artificial antigen-presenting cell, wherein the natural antigen-presenting cell or the artificial antigen-presenting cell comprises a class II major histocompatibility complex of an I-A^{b} haplotype (MHC II I-A^{b}) presenting a peptide comprising a pathogenic T cell epitope of an aquaporin; and b) detecting binding of the autoreactive T cell, if present in the population of CD4+ T cells, to the pathogenic T cell epitope of the aquaporin or activation of the autoreactive T cell in response to the binding of the autoreactive T cell to the pathogenic T cell epitope of the aquaporin.

In certain embodiments, the peptide comprising the pathogenic T cell epitope comprises or consists of amino acids 133-149, 201-220, or 202-218 of aquaporin 4 (AQP4), wherein numbering of amino acid positions is relative to the reference amino acid sequence of SEQ ID NO:8.

In certain embodiments, the peptide comprising the pathogenic T cell epitope comprises or consists of amino acids 174-190 of aquaporin 5 (AQP5), wherein numbering of amino acid positions is relative to the reference amino acid sequence of SEQ ID NO:9

In certain embodiments, the peptide comprising the pathogenic T cell epitope comprises or consists of a sequence selected from the group consisting of SEQ ID NOS: 1-7.

In certain embodiments, the MHC II is a human leukocyte antigen-DR 17 (HLA-DR17) isotype. In some embodiments, the HLA-DR17 isotype is a DRB1*0301 allele.

In certain embodiments, the artificial antigen presenting cell is a MHC II I-A^{b} peptide tetramer.

In certain embodiments, the natural antigen-presenting cell is a dendritic cell.

In certain embodiments, the autoimmune aquaporinopathy is neuromyelitis optica spectrum disorder or Sjögren's syndrome.

In certain embodiments, detecting activation of the autoreactive T cell comprises measuring proliferation, cytokine secretion, or expression of an activation marker.

In certain embodiments, measuring proliferation comprises labeling the CD4⁺ T-cells with ³H-thymidine or a fluorescent tracking dye. Exemplary fluorescent tracking dyes include, without limitation, carboxyfluorescein succinimidyl ester (CFSE) and 5-chloromethylfluorescein diacetate (CMFDA).

In certain embodiments, the autoreactive T cell is a type 1 helper T cell (Tₕ1) or a type 17 helper T cell (Tₕ17). In some embodiments, the Tₕ1 or the Tₕ17 is HLA-DR-restricted.

In certain embodiments, detecting activation of the autoreactive T cell comprises detecting a Tₕ1 immune response or a Tₕ17 immune response. In some embodiments, detecting the Tₕ17 immune response comprises measuring Tₕ17 proliferation, cell surface expression of a transforming growth factor beta receptor, an interleukin 6 receptor, an alpha, interleukin 21 receptor, or an interleukin 23 receptor, or secretion of interleukin 17A (IL-17A), interleukin 17F (IL-17F), interleukin 17AF (IL-17AF), interleukin 21 (IL-21), or interleukin 22 (IL-22), or any combination thereof. In some embodiments, detecting the Th1 immune response comprises measuring Tₕ1 proliferation, cell surface expression of an interleukin 12 receptor beta 2, an interleukin 27 receptor alpha, an interferon gamma receptor 2, an interleukin 18 receptor, a C-C motif chemokine receptor 5 (CCR5), or a C-X-C motif chemokine receptor 3 (CXCR3), or secretion of interferon gamma (IFN-γ), tumor necrosis factor beta (TNF-β), interleukin 2 (IL-2), or interleukin 10 (IL-10), or any combination thereof.

In another aspect, a method of diagnosing and treating an autoimmune aquaporinopathy in a patient is provided, the method comprising: a) obtaining a biological sample comprising a population of CD4+ T cells from the patient; b) contacting the population of CD4⁺ T cells with a natural antigen-presenting cell or an artificial antigen-presenting cell, wherein the natural antigen-presenting cell or the artificial antigen-presenting cell comprises a class II major histocompatibility complex of an I-A^{b} haplotype (MHC II I-A^{b}) presenting a peptide comprising a pathogenic T cell epitope of an aquaporin; c) detecting binding of the autoreactive T cell, if present in the population of CD4+ T cells, to the pathogenic T cell epitope of the aquaporin or activation of the autoreactive T cell in response to the binding of the autoreactive T cell to the pathogenic T cell epitope of the aquaporin, wherein said detecting the binding to the pathogenic T cell epitope or the activation of the autoreactive T cell in response to the binding of the autoreactive T cell to the pathogenic T cell epitope of the aquaporin indicates that the patient has the autoimmune aquaporinopathy; and d) treating the patient for the autoimmune aquaporinopathy if the patient is diagnosed as having the autoimmune aquaporinopathy based on said detecting the binding of the autoreactive T cell to the pathogenic T cell epitope of the aquaporin or the activation of the autoreactive T cell in response to the binding of the autoreactive T cell to the pathogenic T cell epitope of the aquaporin.

In certain embodiments, the biological sample is obtained from blood, bone marrow, spleen, tonsils, or lymph nodes. In some embodiments, the biological sample is peripheral blood mononuclear cells (PBMCs).

Exemplary methods of treating the patient for the autoimmune aquaporinopathy if the patient has a positive diagnosis include, but are not limited to, performing plasma exchange, performing AQP4 or AQP5 immunoglobulin G (IgG) depletion or B cell depletion, performing stem cell transplantation, or administering a steroid, an immunosuppressive agent, an anti-mitotic agent, a complement inhibitor, a calcineurin inhibitor, an inhibitor of guanosine nucleotide biosynthesis, an inhibitor of inosine monophosphate dehydrogenase, or an inhibitor of a folate-dependent enzyme, anti-IL-6 receptor therapy, or a combination thereof.

In another aspect, an artificial antigen-presenting cell for use in diagnosing an autoimmune aquaporinopathy is provided, the artificial antigen-presenting cell comprising a class II major histocompatibility complex of an I-A^{b} haplotype (MHC II I-A^{b}) bound to a peptide comprising a pathogenic T cell epitope of an aquaporin. In certain embodiments, the peptide bound to the MHC II I-A^{b} comprises or consists of amino acids 133-149, 201-220, or 202-218 of aquaporin 4 (AQP4), wherein numbering of amino acid positions is relative to the reference amino acid sequence of SEQ ID NO: 8. In certain embodiments, the peptide bound to the MHC II I-A^{b} comprises or consists of amino acids 174-190 of aquaporin 5 (AQP5), wherein numbering of amino acid positions is relative to the reference amino acid sequence of SEQ ID NO:9. In certain embodiments, the pathogenic T cell epitope comprises or consists of a sequence selected from the group consisting of SEQ ID NOS: 1-7.

In certain embodiments, the MHC II is a human leukocyte antigen-DR 17 (HLA-DR17) isotype.

In certain embodiments, the HLA-DR17 isotype is a DRB1*0301 allele.

In certain embodiments, the artificial antigen presenting cell is a MHC II I-A^{b} peptide tetramer.

In another aspect, a kit is provided, the kit comprising an artificial antigen-presenting cell comprising a class II major histocompatibility complex of an I-A^{b} haplotype (MHC II I-A^{b}) presenting a pathogenic T cell epitope of an aquaporin, described herein, and instructions for detecting an autoreactive T cell associated with an autoimmune aquaporinopathy.

In certain embodiments, the kit further comprises reagents for detecting T cell proliferation, secretion of a cytokine, or expression of an activation marker. In some embodiments, the cytokine is selected from the group consisting of IL-17A, IL-17F, IL-17AF, IL-21, IL-22, IFN-γ, TNF-β, IL-2, and IL-10. In some embodiments, the activation marker is selected from the group consisting of a transforming growth factor beta receptor, an interleukin 6 receptor, an alpha, interleukin 21 receptor, an interleukin 23 receptor, an interleukin 12 receptor beta 2, an interleukin 27 receptor alpha, an interferon gamma receptor 2, an interleukin 18 receptor, a C-C motif chemokine receptor 5 (CCR5), and a C-X-C motif chemokine receptor 3 (CXCR3). In some embodiments, In some embodiments, the reagents comprise ³H-thymidine or a fluorescent tracking dye. In some embodiments, the kit comprises reagents for performing an enzyme-linked immunosorbent spot (ELISpot) assay, an enzyme-linked immunosorbent assay (ELISA) assay, or immunofluorescence.

In another aspect, an in vitro method of diagnosing an autoimmune aquaporinopathy is provided, the method comprising: a) obtaining a biological sample comprising a population of CD4⁺ T cells from the patient; b) contacting the population of CD4⁺ T cells with a natural antigen-presenting cell or an artificial antigen-presenting cell, wherein the natural antigen-presenting cell or the artificial antigen-presenting cell comprises a class II major histocompatibility complex of an I-A^{b} haplotype (MHC II I-A^{b}) presenting a peptide comprising a pathogenic T cell epitope of an aquaporin; and c) detecting binding of the autoreactive T cell, if present in the population of CD4+ T cells, to the pathogenic T cell epitope of the aquaporin or activation of the autoreactive T cell in response to the binding of the autoreactive T cell to the pathogenic T cell epitope of the aquaporin, wherein said detecting the binding to the pathogenic T cell epitope or the activation of the autoreactive T cell in response to the binding of the autoreactive T cell to the pathogenic T cell epitope of the aquaporin indicates that the patient has the autoimmune aquaporinopathy.

In another aspect, a method of monitoring efficacy of a treatment of a patient for an autoimmune aquaporinopathy is provided, the method comprising: obtaining a first biological sample comprising a first population of CD4⁺ T cells from the patient before the patient undergoes the treatment and a second biological sample comprising a second population of CD4⁺ T cells from the patient after the patient undergoes the treatment; contacting the first population of CD4⁺ T cells and the second population of CD4⁺ T cells with a natural antigen-presenting cell or an artificial antigen-presenting cell, wherein the natural antigen-presenting cell or the artificial antigen-presenting cell comprises a class II major histocompatibility complex of an I-A^{b} haplotype (MHC II I-A^{b}) presenting a peptide comprising a pathogenic T cell epitope of an aquaporin; measuring amounts of autoreactive T cells bound to the pathogenic T cell epitope of the aquaporin in the first population of CD4⁺ T cells and the second population of CD4⁺ T cells; and evaluating the efficacy of the treatment, wherein detection of increased amounts of the autoreactive T cells bound to the pathogenic T cell epitope of the aquaporin in the second population of CD4⁺ T cells compared to the first population of CD4⁺ T cells indicate that the patient is worsening or not responding to the treatment, and detection of decreased amounts of the autoreactive T cells bound to the pathogenic T cell epitope of the aquaporin in the second population of CD4⁺ T cells compared to the first population of CD4⁺ T cells indicate that the patient is improving.

In certain embodiments, the method further comprises altering the treatment if the patient is worsening or not responding to the treatment.

In another aspect, a method of monitoring an autoimmune aquaporinopathy in a patient is provided, the method comprising: obtaining a first biological sample comprising a first population of CD4⁺ T cells from the patient at a first time point and a second biological sample comprising a second population of CD4⁺ T cells from the patient later at a second time point; contacting the first population of CD4⁺ T cells and the second population of CD4⁺ T cells with a natural antigen-presenting cell or an artificial antigen-presenting cell, wherein the natural antigen-presenting cell or the artificial antigen-presenting cell comprises a class II major histocompatibility complex of an I-A^{b} haplotype (MHC II I-A^{b}) presenting a peptide comprising a pathogenic T cell epitope of an aquaporin; and measuring amounts of autoreactive T cells bound to the pathogenic T cell epitope of the aquaporin in the first population of CD4⁺ T cells and the second population of CD4⁺ T cells, wherein detection of increased amounts of the autoreactive T cells bound to the pathogenic T cell epitope of the aquaporin in the second population of CD4⁺ T cells compared to the first population of CD4⁺ T cells indicate that the patient is worsening, and detection of decreased amounts of the autoreactive T cells bound to the pathogenic T cell epitope of the aquaporin in the second population of CD4⁺ T cells compared to the first population of CD4⁺ T cells indicate that the patient is improving.

### BRIEF DESCRIPTION OF THE DRAWINGS

FIGS. 1A-1B. AQP4-specific Th17 cells from AQP4-deficient, but not from WT, mice induce CNS autoimmune disease. (FIG. 1A) WT mice are resistant to AQP4 CNS autoimmune disease by direct immunization with AQP4 (13, 14, 17, 18). Donor WT AQP4-specific T cells do not induce CNS autoimmune disease in WT recipient mice. In contrast, donor AQP4-specific T cells from AQP4-deficient mice induce short-lived clinical and histologic CNS autoimmune disease in recipient WT mice. (FIG. 1B) WT mice are susceptible to MOG CNS autoimmune disease (i.e. EAE) induced by immunization with MOG or by adoptive transfer donor WT MOG-specific T cells. Graphics created using BioRender.com.

FIGS. 2A-2F. Pathogenic AQP4 determinants bind I-A^{b} with high affinity and elicit proliferation of AQP4: I-A^{b}-specific TCR-bearing T cells in AQP4-deficient mice. (FIG. 2A) WT or AQP4^{-/-} mice were immunized s.c. with peptides indicated. Peptide-specific proliferation of draining lymph node cells was measured by 3H-thymidine incorporation (mean±SEM, representative of five experiments). (FIG. 2B) Lymph node cells from AQP4 immunized WT or AQP4^{-/-} mice were cultured for 3 days with cognate antigen under Th17 polarizing conditions. AQP4 p133-149- and p202-218-specific Th17 cells from AQP4/donors induce paralysis in WT recipient mice. (FIG. 2C) IEDB, an in silico method for predicting determinants within proteins that bind MHC alleles, was used to identify AQP4 amino acid sequences anticipated to bind I-A^{b} for recognition by AQP4-specific/I-A^{b}-restricted CD4⁺ T cells. 1/IC₅₀ is plotted against the first amino acid for each overlapping AQP4 15 mer. Peaks correspond to increased predicted binding affinity. (FIG. 2D) Purified I-A^{b} molecules were tested biochemically for peptide binding affinity using a competitive binding assay between a radiolabeled high-affinity competitor peptide and unlabeled peptides (22). Lower IC₅₀ values indicate higher binding affinity. Boxed amino acids designate the core binding regions. (FIGS. 2E-2F) Lymph node T cells isolated from WT or AQP4−/− mice immunized with AQP4 p133-149 or p202-218 were stained with I-A^{b}:p133-149 and I-A^{b}:p220-218 tetramers, bead-enriched and analyzed by flow cytometry. FIG. 2E shows representative tetramer staining of bead-enriched lymph node cell samples. FIG. 2F shows frequency of tetramer-binding T cells among CD4⁺ T cells, calculated from enriched and unenriched fractions using counting beads for quantification. Each data point represents one mouse. ****p<0.0001 (Mann Whitney U test).

FIGS. 3A-3C. AQP4-specific TCR repertoire selection differs in AQP4-deficient and WT mice. Draining lymph node cells from AQP4 p133-149-immunized AQP4^{-/-} and WT mice were cultured with p133-149 for 10 days, then restimulated with irradiated splenic APC and p133-149 for an additional 10 days. CD4⁺ T cells were isolated and subjected to scRNA-seq for TCR V (D) J a/b repertoire analysis (3,000-6,000 clonotypes/group/experiment). (FIG. 3A) Pie chart shows the number of individual clonotypes per 10,000 detected in AQP4^{-/-} mice only (black, 54.8%), WT mice only (grey, 44.9%) and those that were common (white, 0.3%) to AQP4/and WT mice. (FIG. 3B) A higher frequency of tet+ (19.3%) than tet (2.3%) TCR a/b clonotypes were shared (red, “public”) by separate AQP4^{-/-} mice. Data represent a total of 16,000 tet+ and tet clonotypes analyzed in two experiments (2 mice/group/experiment). (FIG. 3C) A majority of expanded AQP4-tet⁻ TCR clonotypes and all amplified tet⁺ TCR a/b clonotypes analyzed were not detected among WT AQP4-specific TCR clonotypes. The tet⁺ AQP4 population contained several public TCR clonotypes. A total of 9,700 tet⁻ T cells (3,500 clonotypes) and 6,300 tet⁺ T cells (1,033 clonotypes) are represented.

FIGS. 4A-4B. Negative selection of AQP4 p133-149-reactive T cells is bypassed partially in mice deficient in thymic AQP4 expression. (FIG. 4A) AQP4^{-/-} WT, Foxn1cre-AQP4^{fl/fl}, Aire^{-/-}, or Foxn1cre-Fezf2^{fl/fl} mice were immunized s.c. with peptides indicated. Peptide-specific proliferation of draining lymph node cells was measured by 3H-thymidine incorporation (mean±SEM, representative of five experiments). Comparisons within the five experiments for p133-149 at 10 mg/ml: WT vs. AQP4^{-/-} (p=0.008), WT vs. Foxn1cre-AQP4^{fl/fl} (p=0.008). (FIG. 4B) Ten days after peptide immunization, CD4⁺ T cells from draining lymph nodes were analyzed for I-A^{b}: peptide tetramer binding by flow cytometry. Each data point represents the frequency (mean±SEM) of CD4⁺ T cells bound to cognate antigen-tetramer in a single mouse. Comparisons were calculated by Mann Whitney U Test (p<0.0001****, p<0.001***, p<0.01**, p<0.05*) Comparisons which did not reach statistical significance are not shown. For p133-149: WT vs. Aire^{-/-} (p=0.9), WT vs. Foxn1cre-Fezf2^{fl/fl} (p=0.3). For p202-218: WT vs. Foxn1cre-AQP4^{fl/fl} (p=0.3), WT vs. Aire^{-/-} (p=0.06).

FIGS. 5A-5B. Pathogenic AQP4-specific Th17 cells induce sustained CNS autoimmune disease in T cell-deficient mice. (FIG. 5A) Th17-polarized AQP4 p133-149-specific T cells from AQP4−/− mice were transferred into WT, T cell- and B cell-deficient (T cell-/B cell-, RAG1^{-/-}), T cell-deficient (T cell-, TCRa^{-/-}) and B cell-deficient (B cell-, J_{H}T) recipient mice then scored for clinical disease. Donor Th17-polarized MOG p35-55-specific T cells were transferred into WT recipient mice as a control. Results are representative of four experiments (n=5 per group). (FIG. 5B) Recipient mice were evaluated for histologic evidence of meningeal and CNS parenchymal inflammation (H&E). Results are representative of two experiments (2-4 mice/group/time-point/experiment).

FIGS. 6A-6B. Pathogenic AQP4-specific Th17 cells persist in T cell-deficient mice but not in WT mice. (FIG. 6A) Donor Th17-polarized AQP4 p133-149-specific and MOG p35-55-specific primary T cells from actin-GFP/AQP4^{-/-} mice were transferred into WT or T cell-deficient (TCRa^{-/-}) mice, then examined for recovery in 100 ml blood, axillary and inguinal lymph nodes combined (LN), spleen and CNS at 9 and 21 days by flow cytometry. Cells were gated on single, viable GFP⁺CD4 T cells. Counting beads were used for quantification. (FIG. 6B) AQP4 p133-149 or MOG p35-55-primed Th17-polarized LN cells from AQP4^{-/-} actin-GFP⁺ mice were transferred into WT or TCRa^{-/-} mice, then examined by flow cytometry at the indicated time-points. Data shown for FIG. 6A and FIG. 6B represent a composite from three experiments (n=6 per condition (mean±SEM)). Statistical comparison of surviving numbers of GFP⁺ donor cells was performed by Mann-Whitney U test (p<0.01**).

FIGS. 7A-7B. Reduced survival of pathogenic AQP4-specific Th17 cells WT mice is associated with apoptosis. (FIG. 7A) GFP⁺ T cells were examined for viability and apoptosis by staining with Annexin V and 7-AAD followed by flow cytometry (65, 66). Viable cells are negative for Annexin V and 7-AAD. Apoptotic cells are Annexin V positive, which are further distinguished by early stage (7-AAD negative) and late stage (7-AAD positive). A representative flow staining pattern is shown. The total apoptotic donor populations for CD4⁺GFP⁺ AQP4-specific and MOG-specific T cells are shown (magenta box) with the percentages indicated above. Percentages of late-stage and total apoptotic donor GFP⁺ AQP4-specific T cells were higher than donor GFP⁺MOG-specific T cells in recipient WT mice at day 9 and day 21. (FIG. 7B) Apoptotic GFP⁺CD4⁺ T cells from recipient mice were examined in the spleen and CNS, and counting beads were used for quantification. Data shown represent a composite from three experiments, n=6 per condition (mean±SEM). Statistical analysis for comparison of surviving numbers of GFP⁺ donor cells was performed using a t-test with the Holm-Sidak correction for multiple comparisons (p<0.01**, p<0.001***).

FIG. 8. T cells cross-react with AQP4 and AQP5. AQP4-deficient mice were immunized with 100 μg of AQP5 p174-190. Draining lymph node cells were cultured with AQP4 201-220, then restimulated with the different antigens. Proliferation was measured by ³H-thymidine incorporation of the stimulation to antigen divided by the background stimulation (mean±SEM).

FIGS. 9A-9C. T cells reactive to AQP5, an autoantigen in Sjögren's syndrome also recognize AQP4, the autoantigen in NMO, and can cause CNS autoimmune disease. (FIG. 9A) AQP4-deficient mice were immunized with AQP4 p201-220, AQP5 p174-190 or OVA 323-339. 10-12 days later lymphocytes from draining lymph nodes are removed and cultured with AQP4 201-220 under Th17 polarizing conditions for 72 hours. 2×10⁷ T cells were transferred into TCRα deficient mice, followed by 200 ng Pertussis toxin at days 0 and 2. (FIG. 9B) Th17 polarized T cells from AQP4 and AQP5 primed mice, cultured with AQP4, cause paralysis. (FIG. 9C) Histological analysis showed significantly more meningeal and parenchymal infiltrates in the recipients of the AQP4 (n=8) and AQP5 (n=9) primed donor T cells than OVA (n=10) primed T cells. Statistics performed using ANOVA with Tukey's test for multiple comparisons. ****P<0.0001.

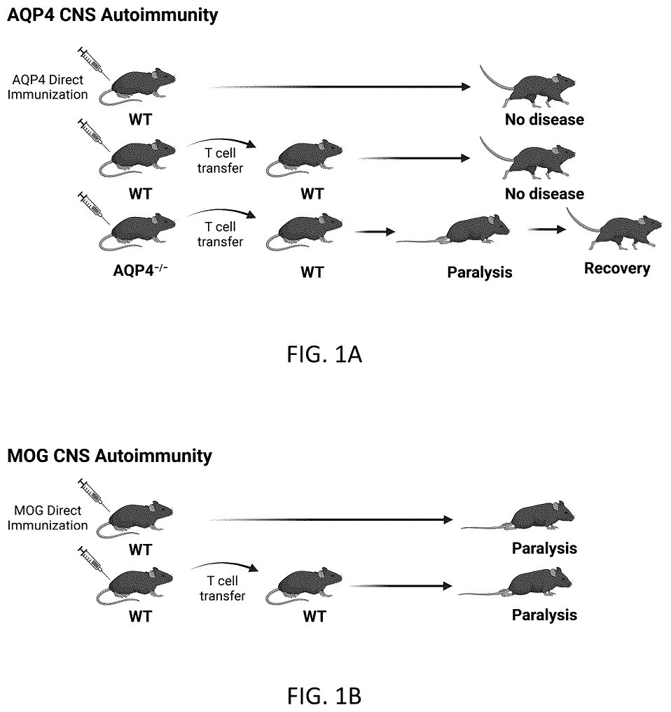

### DETAILED DESCRIPTION OF THE INVENTION

Compositions, methods, and kits are provided for diagnosing autoimmune aquaporinopathy. In particular, cell-based assays are provided for detecting autoreactive T cells that bind to certain pathogenic T cell epitopes of aquaporins. The disclosed methods can be used for diagnosing aquaporinopathies, such as neuromyelitis optica spectrum disorder and Sjögren's syndrome.

Before the present compositions, methods, and kits are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the polypeptide” includes reference to one or more polypeptides and equivalents thereof, e.g., peptides or proteins known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “about,” particularly in reference to a given quantity, is meant to encompass deviations of plus or minus five percent.

The term “T cell” includes all types of immune cells expressing CD3, including T-helper (Tₕ) cells (CD4⁺ T cells), cytotoxic T-cells (CD8⁺ T cells), T-regulatory cells (Tregs), gamma-delta T cells (γδ T cells), and natural killer T cells (NKT cells). CD4⁺ helper T cells include Th1, Th2, Th17, Th9, Tfh, and Th22 subtypes.

The term “autoimmune aquaporinopathy” or “aquaporinopathy”, as used herein, refers to any disease associated with autoreactive T cells and/or autoantibodies against any type of mammalian aquaporin, including aquaporin-0 (AQP0), aquaporin-1 (AQP1), aquaporin-2 (AQP2), aquaporin-3 (AQP3), aquaporin-4 (AQP4), aquaporin-5 (AQP5), aquaporin-6 (AQP6), aquaporin-7 (AQP7), aquaporin-8 (AQP8), aquaporin-9 (AQP9), aquaporin-10 (AQP10), aquaporin-11 (AQP11), or aquaporin-12 (AQP12). Aquaporinopathies include, but are not limited to, neuromyelitis optica spectrum disorder (associated with AQP4 or AQP1 autoimmunity), Sjögren's syndrome (associated with AQP5 autoimmunity), xeropthalmia (associated with AQP1, AQP3, AQP8 or AQP9 autoimmunity), tubulointerstitial nephritis (associated with AQP2 autoimmunity), and autoimmune hemolytic anemia (associated with AQP1 autoimmunity).

The term “biological sample” encompasses a clinical sample, including, but not limited to, a bodily fluid, tissue obtained by surgical resection, tissue obtained by biopsy, cells in culture, cell supernatants, cell lysates, tissue samples, organs, bone marrow, blood, plasma, serum, fine needle aspirate, lymph node aspirate, cystic aspirate, a paracentesis sample, a thoracentesis sample, and the like.

“Isolated” refers to an entity of interest that is in an environment different from that in which it may naturally occur. “Isolated” is meant to include entities that are within samples that are substantially enriched for the entity of interest and/or in which the entity of interest is partially or substantially purified.

“Substantially” or “essentially” means nearly totally or completely, for instance, 95% or greater of some given quantity.

“Substantially purified” generally refers to isolation of a substance (e.g., natural or artificial antigen-presenting cell, cell, compound, small molecule, drug, polynucleotide, protein, polypeptide) such that the substance comprises the majority percent of the sample in which it resides. Typically in a sample, a substantially purified component comprises 50%, preferably 80%-85%, more preferably 90-95% of the sample. Techniques for purifying the substance may include, for example, ion-exchange chromatography, affinity chromatography and sedimentation according to density.

The terms “individual,” “subject,” and “patient” are used interchangeably herein to refer to mammals, including human and non-human mammals such as non-human primates, including chimpanzees and other apes and monkey species; laboratory animals such as mice, rats, rabbits, hamsters, guinea pigs, and chinchillas; domestic animals such as dogs and cats; farm animals such as sheep, goats, pigs, horses and cows. In some cases, the methods of the invention find use in experimental animals, in veterinary application, and in the development of animal models for disease, including, but not limited to, rodents including mice, rats, and hamsters; primates, and transgenic animals.

The term “assaying” is used herein to include the physical steps of manipulating a sample to generate data related to the sample. As will be readily understood by one of ordinary skill in the art, a sample must be “obtained” prior to assaying the sample. Thus, the term “assaying” implies that the sample has been obtained.

The terms “obtained” or “obtaining” as used herein encompass the act of receiving an extracted or isolated sample (e.g., comprising T cells). For example, a testing facility can “obtain” a sample in the mail (or via delivery, etc.) prior to assaying the sample. In some such cases, the sample was “extracted” or “isolated” from an individual by another party prior to mailing (i.e., delivery, transfer, etc.), and then “obtained” by the testing facility upon arrival of the sample. Thus, a testing facility can obtain the sample and then assay the sample, thereby producing data related to the sample.

The terms “obtained” or “obtaining” as used herein can also include the physical extraction or isolation of a sample from a subject. Accordingly, a sample can be isolated from a subject (and thus “obtained”) by the same person or same entity that subsequently assays the sample. When a sample is “extracted” or “isolated” from a first party or entity and then transferred (e.g., delivered, mailed, etc.) to a second party, the sample was “obtained” by the first party (and also “isolated” by the first party), and then subsequently “obtained” (but not “isolated”) by the second party. Accordingly, in some embodiments, the step of obtaining does not comprise the step of isolating a sample.

In some embodiments, the step of obtaining comprises the step of isolating a sample. Methods and protocols for isolating various samples will be known to one of ordinary skill in the art and any convenient method may be used to isolate a sample.

It will be understood by one of ordinary skill in the art that in some cases, it is convenient to wait until multiple samples have been obtained prior to assaying the samples. Accordingly, in some cases an isolated sample is stored until all appropriate samples have been obtained. One of ordinary skill in the art will understand how to appropriately store a variety of different types of samples and any convenient method of storage may be used (e.g., refrigeration) that is appropriate for the particular sample. In some cases, samples are processed immediately or as soon as possible after they are obtained.

“Diagnosis” as used herein generally includes determination as to whether a subject is likely affected by a given disease, disorder or dysfunction. The skilled artisan often makes a diagnosis on the basis of one or more diagnostic indicators (e.g., the presence or absence of an autoreactive antibody which is indicative of the presence or absence of the autoimmune aquaporinopathy).

“Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with autoimmune aquaporinopathy) as well as those in which prevention is desired, those with a genetic predisposition to developing an autoimmune aquaporinopathy, those with increased susceptibility to an autoimmune aquaporinopathy, those suspected of having an autoimmune aquaporinopathy, etc.).

A therapeutic treatment is one in which the subject is inflicted prior to administration and a prophylactic treatment is one in which the subject is not inflicted prior to administration. In some embodiments, the subject has an increased likelihood of becoming inflicted or is suspected of being inflicted prior to treatment. In some embodiments, the subject is suspected of having an increased likelihood of becoming inflicted.

A “therapeutically effective dose”, “therapeutic dose”, or therapeutically effective amount” is an amount sufficient to effect desired clinical results (i.e., achieve therapeutic efficacy). A therapeutically effective dose can be administered in one or more administrations.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer. Both full-length proteins and fragments thereof are encompassed by the definition. The terms also include postexpression modifications of the polypeptide, for example, phosphorylation, glycosylation, acetylation, hydroxylation, oxidation, and the like.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), and any other type of polynucleotide which is an N- or C-glycoside of a purine or pyrimidine base. There is no intended distinction in length between the terms “polynucleotide,” “oligonucleotide,” “nucleic acid” and “nucleic acid molecule,” and these terms are used interchangeably.

“Providing an analysis” is used herein to refer to the delivery of an oral or written analysis (i.e., a document, a report, etc.). A written analysis can be a printed or electronic document. A suitable analysis (e.g., an oral or written report) provides any or all of the following information: identifying information of the subject (name, age, etc.), a description of what type of sample(s) was used and/or how it was used, the technique used to assay the sample, the results of the assay (e.g., whether an autoreactive T cell was detected), the assessment as to whether the individual is determined to have an autoimmune aquaporinopathy, a recommendation for treatment, and/or to continue or alter therapy, a recommended strategy for additional therapy, etc. The report can be in any format including, but not limited to printed information on a suitable medium or substrate (e.g., paper); or electronic format. If in electronic format, the report can be in any computer readable medium, e.g., diskette, compact disk (CD), flash drive, and the like, on which the information has been recorded. In addition, the report may be present as a website address which may be used via the internet to access the information at a remote site.

The terms “specific binding,” “specifically binds,” “selectively binds,” and the like, refer to non-covalent or covalent preferential binding to a molecule relative to other molecules or moieties in a solution or reaction. In some embodiments, the affinity of one molecule for another molecule to which it specifically binds is characterized by a K_{D} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less).

“Affinity” refers to the strength of binding, increased binding affinity being correlated with a lower K_{D}. The binding affinity of a first molecule for a target can be readily determined using conventional techniques, e.g., by competitive enzyme-linked immunosorbent assay (ELISA), flow cytometry, equilibrium dialysis, surface plasmon resonance (SPR), radioimmunoassay; and the like.

### Diagnosing an Autoimmune Aquaporinopathy

Cell-based assays are provided for detecting autoreactive T cells that bind to certain pathogenic T cell epitopes of mammalian aquaporins. The subject methods can be used for detecting autoreactive T cells that bind to pathogenic T cell epitopes of mammalian aquaporins, including aquaporin-0 (AQP0), aquaporin-1 (AQP1), aquaporin-2 (AQP2), aquaporin-3 (AQP3), aquaporin-4 (AQP4), aquaporin-5 (AQP5), aquaporin-6 (AQP6), aquaporin-7 (AQP7), aquaporin-8 (AQP8), aquaporin-9 (AQP9), aquaporin-10 (AQP10), aquaporin-11 (AQP11), and aquaporin-12 (AQP12). Detection of such autoreactive T cells specific for aquaporin autoantigens is useful in diagnosing individuals at risk of developing an autoimmune aquaporinopathy as well as monitoring treatment of patients diagnosed with an autoimmune aquaporinopathy.

Autoimmune aquaporinopathies include any disease associated with autoreactive T cells and/or autoantibodies against any type of mammalian aquaporin, including aquaporin-0 (AQP0), aquaporin-1 (AQP1), aquaporin-2 (AQP2), aquaporin-3 (AQP3), aquaporin-4 (AQP4), aquaporin-5 (AQP5), aquaporin-6 (AQP6), aquaporin-7 (AQP7), aquaporin-8 (AQP8), aquaporin-9 (AQP9), aquaporin-10 (AQP10), aquaporin-11 (AQP11), or aquaporin-12 (AQP12). Aquaporinopathies include, but are not limited to, neuromyelitis optica spectrum disorder (associated with AQP4 or AQP1 autoimmunity), Sjögren's syndrome (associated with AQP5 autoimmunity), xeropthalmia (associated with AQP1, AQP3, AQP8 or AQP9 autoimmunity), tubulointerstitial nephritis (associated with AQP2 autoimmunity), and autoimmune hemolytic anemia (associated with AQP1 autoimmunity).

A biological sample comprising CD4⁺ T cells is obtained from a subject to be diagnosed. The biological sample is typically blood or peripheral blood mononuclear cells (PBMCs) comprising CD4⁺ T cells taken from the subject, but may also be a bone marrow, spleen, tonsil, or lymph node sample, or any other type of biological sample containing CD4⁺ T cells. A “control” sample, as used herein, refers to a sample comprising normal CD4⁺ T cells that are not autoreactive against aquaporins or CD4⁺ T cells from a normal or healthy subject (e.g., an individual known to not have an autoimmune aquaporinopathy). A biological sample comprising CD4⁺ T cells can be obtained from a subject by conventional techniques. For example, blood samples can be obtained by venipuncture, and tissue samples can be obtained by biopsy procedures well known in the art.

The subject methods comprise contacting CD4⁺ T cells in a sample with a natural antigen-presenting cell or an artificial antigen-presenting cell comprising a major histocompatibility complex (MHC) bound to a peptide comprising a pathogenic T cell epitope of an aquaporin. CD4⁺ T cells typically bind to epitopes of 13-17 amino acidic residues presented by major histocompatibility complex class II (MHC II) molecules.

In certain embodiments, the peptide comprising the pathogenic T cell epitope comprises or consists of a sequence corresponding to contiguous amino acids 133-149, 201-220, or 202-218 of the amino acid sequence of aquaporin 4 (AQP4). The foregoing numbering is relative to the amino acid sequence of human AQP4 (SEQ ID NO:8), but it is to be understood that the corresponding amino acid positions of other mammalian aquaporins, including AQP0, AQP1, AQP2, AQP3, AQP5, AQP6, AQP7, AQP8, AQP9, AQP10, AQP11, or AQP12 are also intended to be encompassed by the present invention.

In certain embodiments, the peptide comprising the pathogenic T cell epitope comprises or consists of a sequence corresponding to contiguous amino acids 174-190 of the amino acid sequence of aquaporin 5 (AQP5). The foregoing numbering is relative to the amino acid sequence of human AQP5 (SEQ ID NO:9), but it is to be understood that the corresponding amino acid positions of other mammalian aquaporins, including AQP0, AQP1, AQP2, AQP3, AQP4, AQP6, AQP7, AQP8, AQP9, AQP10, AQP11, or AQP12 are also intended to be encompassed by the present invention.

In certain embodiments, the peptide comprising the pathogenic T cell epitope of an aquaporin comprises or consists of at least 9, at least 10, at least 11, at least 12 at least 13, at least 14, at least 15, at least 16, or at least 17 contiguous amino acids of an amino acid sequence selected from the group consisting of SEQ ID NOS: 1-7. In some embodiments, the peptide comprising the pathogenic T cell epitope of an aquaporin comprises or consists of an amino acid sequence selected from the group consisting of SEQ ID NOS: 1-7.

Examples of natural antigen-presenting cells that can present an antigen of interest (e.g., a peptide comprising a pathogenic T cell epitope of an aquaporin) to T cells include dendritic cells, macrophages, and activated B cells. Alternatively, artificial antigen-presenting cells may be used, such as soluble MHC-multimers or cellular or acellular artificial antigen-presenting cells. MHC-multimers typically range in size from dimers to octamers (tetramers commonly used) and can be used to display class 1 or class 2 MHC bound to an antigen of interest (Hadrup et al. (2009) Nature Methods 6:520-526, Nepom et al. (2003) Antigen 106:1-4, Bakker et al. (2005) Current Opinion in Immunology 17:428-433). Cellular artificial antigen-presenting cells may include cells that have been genetically modified to express T-cell co-stimulatory molecules, MHC alleles and/or cytokines. For example, artificial antigen-presenting cells have been generated from fibroblasts modified to express HLA molecules, the co-stimulatory signal, B7.1, and the cell adhesion molecules, ICAM-1 and LFA-3 (Latouche et al. (2000) Nature Biotechnology 18 (4): 405-409). Acellular antigen-presenting cells comprise biocompatible particles such as microparticles or nanoparticles that carry T cell activating proteins on their surface (Sunshine et al. (2014) Biomaterials 35 (1): 269-277), Perica et al. (2014) Nanomedicine: Nanotechnology, Biology and Medicine 10 (1): 119-129). For a review of artificial antigen-presenting cells, see, e.g., Oelke et al. (2004) Clin. Immunol. 110 (3): 243-251, Wang et al. (2017) Theranostics 7 (14): 3504-3516, Butler et al. (2014) Immunol Rev. 257 (1): 191-209, Eggermont et al. (2014) Trends Biotechnol. 32 (9): 4564-4565, Sunshine et al. (2013) Nanomedicine (Lond) 8 (7): 1173-1189, and Rhodes et al. (2018) Mol. Immunol. 98:13-18; herein incorporated by reference. In certain embodiments, the natural antigen-presenting cell or artificial antigen-presenting cell comprises a class II major histocompatibility complex of an I-A^{b} haplotype (MHC II I-A^{b}). In certain embodiments, the natural antigen-presenting cell or artificial antigen-presenting cell comprises a human leukocyte antigen-DR 17 (HLA-DR17) isotype of MHC II. In some embodiments, the HLA-DR17 isotype is a DRB1*0301 allele.

Binding assays can be used to detect autoreactive T cells that specifically bind to a pathogenic T cell epitope of an aquaporin. Any suitable technique known in the art can be used for detecting such binding such as, but not limited to, competitive enzyme-linked immunosorbent assays (ELISAs), flow cytometry, equilibrium dialysis, or surface plasmon resonance (SPR).

Alternatively or additionally, autoreactive T cells can be detected by their epitope-specific T cell responses to binding to a pathogenic T cell epitope of an aquaporin. Activation of an autoreactive T cell by binding to a pathogenic T cell epitope of an aquaporin can be detected, for example, by measuring cell proliferation, expression of activation markers, or production of effector cytokines, or any combination thereof. In certain embodiments, the subject method comprises detecting a Tₕ17 immune response by measuring Tₕ17 proliferation, cell surface expression of TGF-beta R, IL-6 R alpha, IL-21 R, or IL-23R, or secretion of a cytokine such as IL-17A, IL-17F, IL-17AF, IL-21, or IL-22, or any combination thereof. In certain embodiments, the subject method comprises detecting a Tₕ1 immune response by measuring Tₕ1 proliferation, cell surface expression of IL-12 R beta 2, IL-27 R alpha/WSX-1, IFN-gamma R2, IL-18 R, CCR5, or CXCR3, or secretion of a cytokine such as IFN-γ, TNF-β, IL-2, or IL-10, or any combination thereof.

Various methods, known in the art, can be used to detect T cell activation. For example, flow cytometry can be used to assess cell proliferation and detect activation markers. Lipophilic dyes, such as PKH67 and PKH26 can be used to label the cell membranes of target cells for measuring proliferation of T cells by flow cytometry. Cell proliferation can also be detected and quantified, for example, using a cell counter, measuring uptake of 3H-thymidine, or staining T cells with a fluorescent tracking dye, such as carboxyfluorescein succinimidyl ester (CFSE). In addition, T cell activation can also be detected by immunofluorescent labeling of activation markers such as CD69, HLA-DR, IL2RA (CD25), CD40L, CD137, OX40, PD-L1, 4-1BB IL-12 R beta 2, IL-27 R alpha/WSX-1, IFN-gamma R2, IL-18 R, CCR5, CXCR3, TGF-beta R, IL-6 R alpha, IL-21 R, IL-23 R. Secretion of cytokines can be measured, for example, using a multiplexed enzyme-linked immunosorbent assay (ELISA), an enzyme-linked immunospot (ELISPOT) assay, cytokine capture assays, or intracellular cytokine staining. For a further description of methods of detecting T cell responses, see also, e.g., Tario et al. (2011) Methods Mol Biol 699:119-164, Tario et al. (2018) Methods Mol. Biol. 1678:249-299, Poloni et al. (2023) Immunol. Cell Biol. 101 (6): 491-503, Stempels et al. (2022) J. Immunol. Methods 502:113228, Terren et al. (2020) Methods Enzymol. 631:239-255, Lovelace et al. (2018) Methods Mol. Biol. 1678:151-166, Leehan et al. (2015) Methods Mol. Biol. 1312:427-434, Rodrigues et al. (2017) Cytometry A 91:901-907, Chattopadhyay et al. (2005) Nat Med 11:1113-1117, Frentsch et al. (2005) Nat Med 11:1118-1124; herein incorporated by reference in their entireties.

In some embodiments, the methods described herein are used for monitoring an autoimmune aquaporinopathy in a patient. For example, the amount of autoreactive T cells can be measured in a first biological sample (e.g., blood or PBMCs), obtained from the patient at a first time point, and a second biological sample, obtained from the patient later at a second time point, to determine if the amount of autoreactive T cells is increasing or decreasing over time. In certain embodiments, a method of monitoring an autoimmune aquaporinopathy in a patient is provided, the method comprising: obtaining a first biological sample comprising a first population of CD4⁺ T cells from the patient at a first time point and a second biological sample comprising a second population of CD4+ T cells from the patient later at a second time point; contacting the first population of CD4⁺ T cells and the second population of CD4⁺ T cells with a natural antigen-presenting cell or an artificial antigen-presenting cell, wherein the natural antigen-presenting cell or the artificial antigen-presenting cell comprises a class II major histocompatibility complex of an I-A^{b} haplotype (MHC II I-A^{b}) presenting a peptide comprising a pathogenic T cell epitope of an aquaporin; and measuring amounts of autoreactive T cells bound to the pathogenic T cell epitope of the aquaporin in the first population of CD4⁺ T cells and the second population of CD4⁺ T cells, wherein detection of increased amounts of the autoreactive T cells bound to the pathogenic T cell epitope of the aquaporin in the second population of CD4⁺ T cells compared to the first population of CD4⁺ T cells indicate that the patient is worsening, and detection of decreased amounts of the autoreactive T cells bound to the pathogenic T cell epitope of the aquaporin in the second population of CD4⁺ T cells compared to the first population of CD4⁺ T cells indicate that the patient is improving.

The subject methods may also be used for assaying pre-treatment and post-treatment biological samples obtained from an individual to determine whether the individual is responsive or not responsive to a treatment. In certain embodiments, a method of monitoring efficacy of a treatment of a patient for an autoimmune aquaporinopathy is provided, the method comprising: obtaining a first biological sample comprising a first population of CD4⁺ T cells from the patient before the patient undergoes the treatment and a second biological sample comprising a second population of CD4⁺ T cells from the patient after the patient undergoes the treatment; contacting the first population of CD4⁺ T cells and the second population of CD4⁺ T cells with a natural antigen-presenting cell or an artificial antigen-presenting cell, wherein the natural antigen-presenting cell or the artificial antigen-presenting cell comprises a class II major histocompatibility complex of an I-A^{b} haplotype (MHC II I-A^{b}) presenting a peptide comprising a pathogenic T cell epitope of an aquaporin; measuring amounts of autoreactive T cells bound to the pathogenic T cell epitope of the aquaporin in the first population of CD4⁺ T cells and the second population of CD4⁺ T cells; and evaluating the efficacy of the treatment, wherein detection of increased amounts of the autoreactive T cells bound to the pathogenic T cell epitope of the aquaporin in the second population of CD4⁺ T cells compared to the first population of CD4⁺ T cells indicate that the patient is worsening or not responding to the treatment, and detection of decreased amounts of the autoreactive T cells bound to the pathogenic T cell epitope of the aquaporin in the second population of CD4⁺ T cells compared to the first population of CD4⁺ T cells indicate that the patient is improving. In certain embodiments, the method further comprises altering the treatment if the patient is worsening or not responding to the treatment.

The method may further comprise determining an appropriate treatment regimen for a patient and, in particular, whether a patient should be treated for an autoimmune aquaporinopathy. For example, a patient is selected for treatment for an autoimmune aquaporinopathy if the patient has a positive diagnosis for an autoimmune aquaporinopathy based on detection of binding of an autoreactive T cell to a pathogenic T cell epitope of the aquaporin or activation of the autoreactive T cell in response to the binding of the autoreactive T cell to the pathogenic T cell epitope of the aquaporin according to the methods described herein. Treatment may comprise, performing plasma exchange, performing AQP4 or AQP5 immunoglobulin G (IgG) depletion or B cell depletion, performing stem cell transplantation, or administering a steroid such as methylprednisolone or prednisone, an anti-mitotic agent such as cyclophosphamide or mitoxantrone, intravenous immunoglobulin (IVIG), a complement inhibitor such as eculizumab, a calcineurin inhibitor such as cyclosporine A, an inhibitor of guanosine nucleotide biosynthesis such as azathioprine, an inhibitor of inosine monophosphate dehydrogenase such as mycophenolate mofetil, or an inhibitor of a folate-dependent enzyme such as methotrexate, anti-IL-6 receptor therapeutic agents such as tocilizumab or satralizumab, or a combination thereof. Methylprednisolone and plasma exchange are commonly administered as the first therapy and for acute attacks. In more severe cases, cyclophosphamide is typically added to the treatment. Rituximab, an anti-CD20 monoclonal antibody, or inebilizumab, an anti-CD19 monoclonal antibody, can be used to deplete B cells. Mycophenolate mofetil may be added as an immunosuppressive agent. In addition, treatment with azathioprine, a guanosine nucleotide biosynthesis inhibitor, eculizumab, a monoclonal antibody against the C5 complement protein, tocilizumab, a monoclonal antibody against the interleukin 6 receptor, methotrexate, a folate-dependent enzyme inhibitor, mitoxantrone, a topoisomerase II inhibitor and an anti-mitotic agent, and oral corticosteroids may be added to reduce the relapse rate. Autologous hemopoietic stem cell transplantation may be administered to patients who do not respond to conventional therapy.

In some cases, the diagnostic methods described herein may be used by themselves or combined with screening for autoantibodies (e.g., IgG against AQP4, AQP5, or myelin oligodendrocyte glycoprotein) and/or medical imaging (e.g., detecting lesions in the brain, optic nerve, or spinal cord) to confirm the diagnosis and further evaluate the severity and extent of disease. Medical imaging can be used for example to detect inflammatory demyelination, necrotic damage of white and grey matter, and inflammatory lesions associated with the disease. Exemplary medical imaging techniques include, without limitation, magnetic resonance imaging (MRI), diffusion tensor imaging (DTI), positron emission tomography (PET), single photon emission computed tomography (SPECT), computed tomography (CT), ultrasound imaging (UI), optical imaging (OI), photoacoustic imaging (PI), fluoroscopy, or fluorescence imaging.

The amount of an autoreactive T cell in a pre-treatment biological sample can be referred to as a “pre-treatment value” because the first biological sample is isolated from the individual prior to the administration of the therapy (i.e., “pre-treatment”). The level of an autoreactive T cell in the pre-treatment biological sample can also be referred to as a “baseline value” because this value is the value to which “post-treatment” values are compared. In some cases, the baseline value (i.e., “pre-treatment value”) is determined by determining the level of an autoreactive T cell in multiple (i.e., more than one, e.g., two or more, three or more, for or more, five or more, etc.) pre-treatment biological samples. In some cases, the multiple pre-treatment biological samples are isolated from an individual at different time points in order to assess natural fluctuations in autoreactive T cell levels prior to treatment. As such, in some cases, one or more (e.g., two or more, three or more, for or more, five or more, etc.) pre-treatment biological samples are isolated from the individual. In some embodiments, all of the pre-treatment biological samples will be the same type of biological sample (e.g., a blood sample). In some cases, two or more pre-treatment biological samples are pooled prior to determining the level of the autoreactive T cell in the biological samples. In some cases, the level of the autoreactive T cell is determined separately for two or more pre-treatment biological samples and a “pre-treatment value” is calculated by averaging the separate measurements.

A post-treatment biological sample is isolated from an individual after the administration of a therapy. Thus, the level of an autoreactive T cell in a post-treatment sample can be referred to as a “post-treatment value”. In some embodiments, the level of an autoreactive T cell is measured in additional post-treatment biological samples (e.g., a second, third, fourth, fifth, etc. post-treatment biological sample). Because additional post-treatment biological samples are isolated from the individual after the administration of a treatment, the levels of an autoreactive T cell in the additional biological samples can also be referred to as “post-treatment values.”

The term “responsive” as used herein means that the treatment is having the desired effect such as reducing the levels of autoreactive T cells and/or autoantibodies against an aquaporin, reducing inflammatory damage and lesions in the brain and spinal cord, preventing, delaying, or reducing optic neuritis, myelitis, and/or loss of vision. When the individual does not improve in response to the treatment, it may be desirable to seek a different therapy or treatment regime for the individual.

The determination that an individual has an aquaporinopathy is an active clinical application based on the correlation between amounts of an autoreactive T cell and disease. For example, “determining” requires the active step of reviewing the data, which is produced during the active assaying step(s), and determining whether an individual does or does not have an aquaporinopathy or risk of developing an aquaporinopathy. Additionally, in some cases, a decision is made to proceed with a current treatment (i.e., therapy), or instead to alter the treatment. In some cases, the subject methods include the step of continuing therapy or altering therapy.

The term “continue treatment” (i.e., continue therapy) is used herein to mean that the current course of treatment (e.g., continued administration of a therapy) is to continue. If the current course of treatment is not effective in treating an autoimmune aquaporinopathy, the treatment may be altered. “Altering therapy” is used herein to mean “discontinuing therapy” or “changing the therapy” (e.g., changing the type of treatment, changing the particular dose and/or frequency of administration of medication, e.g., increasing the dose and/or frequency). In some cases, therapy can be altered until the individual is deemed to be responsive. In some embodiments, altering therapy means changing which type of treatment is administered, discontinuing a particular treatment altogether, etc.

As a non-limiting illustrative example, a patient may be initially treated for an autoimmune aquaporinopathy by performing plasma exchange and administering methylpredisolone. Then to “continue treatment” would be to continue with this type of treatment. If the current course of treatment is not effective, the treatment may be altered, e.g., switching treatment to a steroid or increasing the dose or frequency of administration of the steroid, or changing to a different type of treatment such as administering an anti-mitotic agent such as cyclophosphamide.

In other words, the amount of autoreactive T cells may be monitored in order to determine when to continue therapy and/or when to alter therapy. As such, a post-treatment biological sample can be isolated after any of the administrations and the biological sample can be assayed to determine the amount of autoreactive T cells. Accordingly, the subject methods can be used to determine whether an individual being treated for an autoimmune aquaporinopathy is responsive or is maintaining responsiveness to a treatment.

The therapy can be administered to an individual any time after a pre-treatment biological sample is isolated from the individual, but it is preferable for the therapy to be administered simultaneous with or as soon as possible (e.g., about 7 days or less, about 3 days or less, e.g., 2 days or less, 36 hours or less, 1 day or less, 20 hours or less, 18 hours or less, 12 hours or less, 9 hours or less, 6 hours or less, 3 hours or less, 2.5 hours or less, 2 hours or less, 1.5 hours or less, 1 hour or less, 45 minutes or less, 30 minutes or less, 20 minutes or less, 15 minutes or less, 10 minutes or less, 5 minutes or less, 2 minutes or less, or 1 minute or less) after a pre-treatment biological sample is isolated (or, when multiple pre-treatment biological samples are isolated, after the final pre-treatment biological sample is isolated).

In some cases, more than one type of therapy may be administered to the individual. For example, a subject who has an autoimmune aquaporinopathy may be treated with a corticosteroid and an anti-mitotic agent or an inhibitor of guanosine nucleotide biosynthesis. A subject who has more severe disease or who is at high risk of disease progression, may be treated more aggressively. For example, treatment of a high-risk patient may include, without limitation, administering cyclophosphamide, rituximab, inebilizumab, mycophenolate mofetil, azathioprine, eculizumab, tocilizumab, methotrexate, mitoxantrone, or performing autologous hemopoietic stem cell transplantation, or a combination thereof.

In some embodiments, the subject methods include providing an analysis (e.g., an oral or written report) having any or all of the following information: identifying information of the subject (name, age, etc.), a description of what type of sample(s) was used and/or how it was used, the technique used to assay the sample, the results of the assay (e.g., whether an autoreactive T cell was detected), the assessment as to whether the individual is determined to have an autoimmune aquaporinopathy, a recommendation for treatment, and/or to continue or alter therapy, a recommended strategy for additional therapy, etc. As described above, an analysis can be an oral or written report (e.g., written or electronic document). The analysis can be provided to the subject, to the subject's physician, to a testing facility, etc. The analysis can also be accessible as a website address via the internet. In some such cases, the analysis can be accessible by multiple different entities (e.g., the subject, the subject's physician, a testing facility, etc.).

### Kits

Also provided are kits for use in performing the methods described herein for detecting autoreactive T cells that bind to a pathogenic T cell epitope of an aquaporin. The kit may comprise a container for holding a biological sample such as blood or PBMCs collected from a patient. The kit may also comprise a natural antigen-presenting cell or an artificial antigen-presenting cell comprising a class II major histocompatibility complex of an I-A° haplotype (MHC II I-A^{b}). In certain embodiments, the MHC II is a human leukocyte antigen-DR 17 (HLA-DR17) isotype. In some embodiments, the HLA-DR17 isotype is a DRB1*0301 allele. In certain embodiments, the kit provides an MHC II I-A^{b} peptide tetramer as an artificial antigen presenting cell. In certain embodiments, the kit provides a dendritic cell as a natural antigen-presenting cell. The kit may further comprise a peptide comprising a pathogenic T cell epitope of an aquaporin, either bound to the MHC II I-A^{b} or provided separately. In certain embodiments, the peptide comprising the pathogenic T cell epitope comprises or consists of amino acids 133-149 or 202-218 of aquaporin 4 (AQP4), wherein numbering of amino acid positions is relative to the reference amino acid sequence of SEQ ID NO:8. In certain embodiments, the peptide comprising the pathogenic T cell epitope comprises or consists of amino acids 174-190 of aquaporin 5 (AQP5), wherein numbering of amino acid positions is relative to the reference amino acid sequence of SEQ ID NO:9. In certain embodiments, the peptide comprising the pathogenic T cell epitope comprises or consists of a sequence selected from the group consisting of SEQ ID NOS: 1-7. The kit may further comprise one or more reagents for detecting T cell proliferation (e.g., ³H-thymidine, fluorescent tracking dye such as CFSE), activation markers (e.g., reagents for performing immunofluorescence such as fluorescently labeled antibodies specific for activation markers), or secretion of cytokines (e.g., reagents for performing an ELISA, ELISPOT, cytokine capture assays, intracellular cytokine staining, immunofluorescence). In addition, the kit may contain media for culturing T cells, buffers, control reference samples or standards, and the like.

Such kits generally will comprise, in suitable means, distinct containers for each individual reagent or solution. Suitable containers for the compositions include, for example, bottles, vials, syringes, and test tubes. Containers can be formed from a variety of materials, including glass or plastic. A container may have a sterile access port (for example, the container may be a vial having a stopper pierceable by a hypodermic injection needle).

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), DVD, flash drive, and the like, on which the information has been recorded. Yet another form of these instructions that may be present is a website address which may be used via the internet to access the information at a removed site.

Such kits can be used for diagnosing individuals at risk of developing an autoimmune aquaporinopathy as well as monitoring treatment of patients diagnosed with an autoimmune aquaporinopathy.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-75 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A method of detecting an autoreactive T cell associated with an autoimmune aquaporinopathy, the method comprising:
a) contacting a population of CD4⁺ T cells with a natural antigen-presenting cell or an artificial antigen-presenting cell, wherein the natural antigen-presenting cell or the artificial antigen-presenting cell comprises a class II major histocompatibility complex of an I-A^{b} haplotype (MHC II I-A^{b}) presenting a peptide comprising a pathogenic T cell epitope of an aquaporin; and
b) detecting binding of the autoreactive T cell, if present in the population of CD4⁺ T cells, to the pathogenic T cell epitope of the aquaporin or activation of the autoreactive T cell in response to the binding of the autoreactive T cell to the pathogenic T cell epitope of the aquaporin.

2. The method of aspect 1, wherein the peptide comprising the pathogenic T cell epitope comprises or consists of amino acids 133-149, 201-220, or 202-218 of aquaporin 4 (AQP4), wherein numbering of amino acid positions is relative to the reference amino acid sequence of SEQ ID NO:8.

3. The method of aspect 1, wherein the peptide comprising the pathogenic T cell epitope comprises or consists of amino acids 174-190 of aquaporin 5 (AQP5), wherein numbering of amino acid positions is relative to the reference amino acid sequence of SEQ ID NO:9.

4. The method of aspect 1, wherein the peptide comprising the pathogenic T cell epitope comprises or consists of a sequence selected from the group consisting of SEQ ID NOS: 1-7.

5. The method of any one of aspects 1-4, wherein the MHC II is a human leukocyte antigen-DR 17 (HLA-DR17) isotype.

6. The method of aspect 5, wherein the HLA-DR17 isotype is a DRB1*0301 allele.

7. The method of any one of aspects 1-6, wherein the artificial antigen presenting cell is a MHC II I-A^{b} peptide tetramer.

8. The method of any one of aspects 1-6, wherein the natural antigen-presenting cell is a dendritic cell.

9. The method of any one of aspects 1-8, wherein the autoimmune aquaporinopathy is neuromyelitis optica spectrum disorder or Sjögren's syndrome.

10. The method of any one of aspects 1-9, wherein said detecting activation of the autoreactive T cell comprises measuring proliferation, cytokine secretion, or expression of an activation marker.

11. The method of aspect 10, wherein said measuring proliferation comprises labeling the CD4⁺ T-cells with ³H-thymidine or a fluorescent tracking dye.

12. The method of aspect 11, wherein the fluorescent tracking dye is carboxyfluorescein succinimidyl ester (CFSE) or 5-chloromethylfluorescein diacetate (CMFDA).

13. The method of any one of aspects 1-12, wherein the autoreactive T cell is a type 1 helper T cell (Tₕ1) or a type 17 helper T cell (Tₕ17).

14. The method of aspect 13, wherein the Tₕ1 or the Tₕ17 is HLA-DR-restricted.

15. The method of aspect 13 or 14, wherein said detecting activation of the autoreactive T cell comprises detecting a Tₕ1 immune response or a Tₕ17 immune response, or a combination thereof.

16. The method of aspect 15, wherein said detecting the Tₕ17 immune response comprises measuring Tₕ17 proliferation, cell surface expression of a transforming growth factor beta receptor, an interleukin 6 receptor, an alpha, interleukin 21 receptor, or an interleukin 23 receptor, or secretion of interleukin 17A (IL-17A), interleukin 17F (IL-17F), interleukin 17AF (IL-17AF), interleukin 21 (IL-21), or interleukin 22 (IL-22), or any combination thereof.

17. The method of aspect 15, wherein said detecting the Tₕ1 immune response comprises measuring Tₕ1 proliferation, cell surface expression of an interleukin 12 receptor beta 2, an interleukin 27 receptor alpha, an interferon gamma receptor 2, an interleukin 18 receptor, a C-C motif chemokine receptor 5 (CCR5), or a C-X-C motif chemokine receptor 3 (CXCR3), or secretion of interferon gamma (IFN-γ), tumor necrosis factor beta (TNF-β), interleukin 2 (IL-2), or interleukin 10 (IL-10), or any combination thereof.

18. A method of diagnosing and treating an autoimmune aquaporinopathy in a patient, the method comprising:
a) obtaining a biological sample comprising a population of CD4⁺ T cells from the patient;
b) contacting the population of CD4⁺ T cells with a natural antigen-presenting cell or an artificial antigen-presenting cell, wherein the natural antigen-presenting cell or the artificial antigen-presenting cell comprises a class II major histocompatibility complex of an I-A^{b} haplotype (MHC II I-A^{b}) presenting a peptide comprising a pathogenic T cell epitope of an aquaporin;
c) detecting binding of the autoreactive T cell, if present in the population of CD4+ T cells, to the pathogenic T cell epitope of the aquaporin or activation of the autoreactive T cell in response to the binding of the autoreactive T cell to the pathogenic T cell epitope of the aquaporin, wherein said detecting the binding to the pathogenic T cell epitope or the activation of the autoreactive T cell in response to the binding of the autoreactive T cell to the pathogenic T cell epitope of the aquaporin indicates that the patient has the autoimmune aquaporinopathy; and
d) treating the patient for the autoimmune aquaporinopathy if the patient is diagnosed as having the autoimmune aquaporinopathy based on said detecting the binding of the autoreactive T cell to the pathogenic T cell epitope of the aquaporin or the activation of the autoreactive T cell in response to the binding of the autoreactive T cell to the pathogenic T cell epitope of the aquaporin.

19. The method of aspect 18, wherein the peptide comprising the pathogenic T cell epitope comprises or consists of amino acids 133-149, 201-220, or 202-218 of aquaporin 4 (AQP4), wherein numbering of amino acid positions is relative to the reference amino acid sequence of SEQ ID NO: 8.

20. The method of aspect 18, wherein the peptide comprising the pathogenic T cell epitope comprises or consists of amino acids 174-190 of aquaporin 5 (AQP5), wherein numbering of amino acid positions is relative to the reference amino acid sequence of SEQ ID NO:9.

21. The method of aspect 18, wherein the peptide comprising the pathogenic T cell epitope comprises or consists of a sequence selected from the group consisting of SEQ ID NOS: 1-7.

22. The method of any one of aspects 18-21, wherein the MHC II is a human leukocyte antigen-DR 17 (HLA-DR17) isotype.

23. The method of aspect 22, wherein the HLA-DR17 isotype is a DRB1*0301 allele.

24. The method of any one of aspects 18-23, wherein the artificial antigen presenting cell is a MHC II I-A^{b} peptide tetramer.

25. The method of any one of aspects 18-23, wherein the natural antigen-presenting cell is a dendritic cell.

26. The method of any one of aspects 18-25, wherein the autoimmune aquaporinopathy is neuromyelitis optica spectrum disorder or Sjögren's syndrome.

27. The method of any one of aspects 18-26, wherein said detecting activation of the autoreactive T cell comprises measuring proliferation, cytokine secretion, or expression of an activation marker.

28. The method of aspect 27, wherein said measuring proliferation comprises labeling the CD4⁺ T-cells with 3H-thymidine or a fluorescent tracking dye.

29. The method of aspect 28, wherein the fluorescent tracking dye is carboxyfluorescein succinimidyl ester (CFSE) or 5-chloromethylfluorescein diacetate (CMFDA).

30. The method of any one of aspects 18-29, wherein the autoreactive T cell is a type 1 helper T cell (Tₕ1) or a type 17 helper T cell (Tₕ17).

31. The method of aspect 30, wherein the Tₕ1 or the Tₕ17 is HLA-DR-restricted.

32. The method of aspect 30 or 31, wherein said detecting activation of the autoreactive T cell comprises detecting a Tₕ1 immune response or a Tₕ17 immune response, or a combination thereof.

33. The method of aspect 32, wherein said detecting the Tₕ17 immune response comprises measuring Tₕ17 proliferation, cell surface expression of a transforming growth factor beta receptor, an interleukin 6 receptor, an alpha, interleukin 21 receptor, or an interleukin 23 receptor, or secretion of interleukin 17A (IL-17A), interleukin 17F (IL-17F), interleukin 17AF (IL-17AF), interleukin 21 (IL-21), or interleukin 22 (IL-22), or any combination thereof.

34. The method of aspect 32 or 33, wherein said detecting the Tₕ1 immune response comprises measuring Tₕ1 proliferation, cell surface expression of an interleukin 12 receptor beta 2, an interleukin 27 receptor alpha, an interferon gamma receptor 2, an interleukin 18 receptor, a C-C motif chemokine receptor 5 (CCR5), or a C-X-C motif chemokine receptor 3 (CXCR3), or secretion of interferon gamma (IFN-γ), tumor necrosis factor beta (TNF-β), interleukin 2 (IL-2), or interleukin 10 (IL-10), or any combination thereof.

35. The method of any one of aspects 18-34, wherein the biological sample is obtained from blood, bone marrow, spleen, tonsils, or lymph nodes.

36. The method of any one of aspects 18-35, wherein the biological sample is blood or peripheral blood mononuclear cells (PBMCs).

37. The method of any one of aspects 18-36, further comprising detecting an antibody that specifically binds to the aquaporin in the biological sample, wherein said detecting the antibody that specifically binds to the aquaporin in combination with said detecting the binding of the autoreactive T cell to the pathogenic T cell epitope of the aquaporin or the activation of the autoreactive T cell in response to the binding of the autoreactive T cell to the pathogenic T cell epitope of the aquaporin indicate that the patient has the autoimmune aquaporinopathy.

38. The method of any one of aspects 18-37, wherein said treating the patient for the autoimmune aquaporinopathy comprises performing plasma exchange, performing AQP4 or AQP5 immunoglobulin G (IgG) depletion or B cell depletion, performing stem cell transplantation, or administering a steroid, an immunosuppressive agent, an anti-mitotic agent, intravenous immunoglobulin (IVIG), a complement inhibitor, a calcineurin inhibitor, an inhibitor of guanosine nucleotide biosynthesis, an inhibitor of inosine monophosphate dehydrogenase, or an inhibitor of a folate-dependent enzyme, anti-IL-6 receptor therapy, or a combination thereof.

39. The method of aspect 38, wherein the steroid is methylprednisolone or prednisone.

40. The method of aspect 38 or 39, wherein the anti-mitotic agent is cyclophosphamide or mitoxantrone.

41. The method of any one of aspects 38-40, wherein said performing the B cell depletion comprises administering rituximab to the patient.

42. An artificial antigen-presenting cell comprising a class II major histocompatibility complex of an I-A^{b} haplotype (MHC II I-A^{b}) bound to a peptide comprising a pathogenic T cell epitope of an aquaporin.

43. The artificial antigen-presenting cell of aspect 42, wherein the peptide comprising the pathogenic T cell epitope comprises or consists of amino acids 133-149, 201-220, or 202-218 of aquaporin 4 (AQP4), wherein numbering of amino acid positions is relative to the reference amino acid sequence of SEQ ID NO:8.

44. The artificial antigen-presenting cell of aspect 42, wherein the peptide comprising the pathogenic T cell epitope comprises or consists of amino acids 174-190 of aquaporin 5 (AQP5), wherein numbering of amino acid positions is relative to the reference amino acid sequence of SEQ ID NO: 9.

45. The artificial antigen-presenting cell of aspect 42, wherein the peptide comprising the pathogenic T cell epitope comprises or consists of a sequence selected from the group consisting of SEQ ID NOS: 1-7.

46. The artificial antigen-presenting cell of any one of aspects 42-45, wherein the MHC II is a human leukocyte antigen-DR 17 (HLA-DR17) isotype.

47. The artificial antigen-presenting cell of aspect 46, wherein the HLA-DR17 isotype is a DRB1*0301 allele.

48. The artificial antigen-presenting cell of any one of aspects 42-47, wherein the artificial antigen presenting cell is a MHC II I-A^{b} peptide tetramer.

49. The artificial antigen-presenting cell of any one of aspects 42-48 for use in diagnosing an autoimmune aquaporinopathy.

50. A kit comprising the artificial antigen-presenting cell of any one of aspects 42-49 and instructions for detecting an autoreactive T cell associated with an autoimmune aquaporinopathy.

51. The kit of aspect 50, further comprising reagents for detecting T cell proliferation, secretion of a cytokine, or expression of an activation marker, or a combination thereof.

52. The kit of aspect 51, wherein the cytokine is selected from the group consisting of interleukin 17A (IL-17A), interleukin 17F (IL-17F), interleukin 17AF (IL-17AF), interleukin 21 (IL-21), interleukin 22 (IL-22), interferon gamma (IFN-γ), tumor necrosis factor beta (TNF-β), interleukin 2 (IL-2), and interleukin 10 (IL-10).

53. The kit of aspect 51 or 52, wherein the activation marker is selected from the group consisting of a transforming growth factor beta receptor, an interleukin 6 receptor, an alpha, interleukin 21 receptor, an interleukin 23 receptor, an interleukin 12 receptor beta 2, an interleukin 27 receptor alpha, an interferon gamma receptor 2, an interleukin 18 receptor, a C-C motif chemokine receptor 5 (CCR5), and a C-X-C motif chemokine receptor 3 (CXCR3).

54. The kit of any one of aspects 51-53, wherein the reagents comprise reagents for performing an enzyme-linked immunosorbent assay, an enzyme-linked immunosorbent assay (ELISA) assay, or an immunofluorescent assay.

55. The kit of any one of aspects 51-54, wherein the reagents comprise 3H-thymidine or a fluorescent tracking dye.

56. An in vitro method of diagnosing an autoimmune aquaporinopathy, the method comprising:
a) obtaining a biological sample comprising a population of CD4⁺ T cells from the patient;
b) contacting the population of CD4⁺ T cells with a natural antigen-presenting cell or an artificial antigen-presenting cell, wherein the natural antigen-presenting cell or the artificial antigen-presenting cell comprises a class II major histocompatibility complex of an I-A^{b} haplotype (MHC II I-A^{b}) presenting a peptide comprising a pathogenic T cell epitope of an aquaporin; and
c) detecting binding of the autoreactive T cell, if present in the population of CD4+ T cells, to the pathogenic T cell epitope of the aquaporin or activation of the autoreactive T cell in response to the binding of the autoreactive T cell to the pathogenic T cell epitope of the aquaporin, wherein said detecting the binding to the pathogenic T cell epitope or the activation of the autoreactive T cell in response to the binding of the autoreactive T cell to the pathogenic T cell epitope of the aquaporin indicates that the patient has the autoimmune aquaporinopathy.

57. A method of monitoring efficacy of a treatment of a patient for an autoimmune aquaporinopathy, the method comprising:
obtaining a first biological sample comprising a first population of CD4⁺ T cells from the patient before the patient undergoes the treatment and a second biological sample comprising a second population of CD4⁺ T cells from the patient after the patient undergoes the treatment;
contacting the first population of CD4⁺ T cells and the second population of CD4⁺ T cells with a natural antigen-presenting cell or an artificial antigen-presenting cell, wherein the natural antigen-presenting cell or the artificial antigen-presenting cell comprises a class II major histocompatibility complex of an I-A^{b} haplotype (MHC II I-A^{b}) presenting a peptide comprising a pathogenic T cell epitope of an aquaporin;
measuring amounts of autoreactive T cells bound to the pathogenic T cell epitope of the aquaporin in the first population of CD4⁺ T cells and the second population of CD4⁺ T cells; and
evaluating the efficacy of the treatment, wherein detection of increased amounts of the autoreactive T cells bound to the pathogenic T cell epitope of the aquaporin in the second population of CD4⁺ T cells compared to the first population of CD4⁺ T cells indicate that the patient is worsening or not responding to the treatment, and detection of decreased amounts of the autoreactive T cells bound to the pathogenic T cell epitope of the aquaporin in the second population of CD4⁺ T cells compared to the first population of CD4⁺ T cells indicate that the patient is improving.

58. The method of aspect 57, further comprising altering the treatment if the patient is worsening or not responding to the treatment.

59. The method of aspect 57 or 58, wherein the peptide comprising the pathogenic T cell epitope comprises or consists of amino acids 133-149, 201-220, or 202-218 of aquaporin 4 (AQP4), wherein numbering of amino acid positions is relative to the reference amino acid sequence of SEQ ID NO: 8.

60. The method of aspect 57 or 58, wherein the peptide comprising the pathogenic T cell epitope comprises or consists of amino acids 174-190 of aquaporin 5 (AQP5), wherein numbering of amino acid positions is relative to the reference amino acid sequence of SEQ ID NO:9.

61. The method of aspect 57 or 58, wherein the peptide comprising the pathogenic T cell epitope comprises or consists of a sequence selected from the group consisting of SEQ ID NOS: 1-7.

62. The method of any one of aspects 57-61, wherein the MHC II is a human leukocyte antigen-DR 17 (HLA-DR17) isotype.

63. The method of aspect 62, wherein the HLA-DR17 isotype is a DRB1*0301 allele.

64. The method of any one of aspects 57-63, wherein the artificial antigen presenting cell is a MHC II I-A^{b} peptide tetramer.

65. The method of any one of aspects 57-63, wherein the natural antigen-presenting cell is a dendritic cell.

66. The method of any one of aspects 57-65, wherein the autoimmune aquaporinopathy is neuromyelitis optica spectrum disorder or Sjögren's syndrome.

67. A method of monitoring an autoimmune aquaporinopathy in a patient, the method comprising:
obtaining a first biological sample comprising a first population of CD4⁺ T cells from the patient at a first time point and a second biological sample comprising a second population of CD4⁺ T cells from the patient later at a second time point;
contacting the first population of CD4⁺ T cells and the second population of CD4⁺ T cells with a natural antigen-presenting cell or an artificial antigen-presenting cell, wherein the natural antigen-presenting cell or the artificial antigen-presenting cell comprises a class II major histocompatibility complex of an I-A^{b} haplotype (MHC II I-A^{b}) presenting a peptide comprising a pathogenic T cell epitope of an aquaporin; and
measuring amounts of autoreactive T cells bound to the pathogenic T cell epitope of the aquaporin in the first population of CD4⁺ T cells and the second population of CD4⁺ T cells, wherein detection of increased amounts of the autoreactive T cells bound to the pathogenic T cell epitope of the aquaporin in the second population of CD4⁺ T cells compared to the first population of CD4⁺ T cells indicate that the patient is worsening, and detection of decreased amounts of the autoreactive T cells bound to the pathogenic T cell epitope of the aquaporin in the second population of CD4⁺ T cells compared to the first population of CD4⁺ T cells indicate that the patient is improving.

68. The method of aspect 67, wherein the peptide comprising the pathogenic T cell epitope comprises or consists of amino acids 133-149, 201-220, or 202-218 of aquaporin 4 (AQP4), wherein numbering of amino acid positions is relative to the reference amino acid sequence of SEQ ID NO: 8.

69. The method of aspect 67 or 68, wherein the peptide comprising the pathogenic T cell epitope comprises or consists of amino acids 174-190 of aquaporin 5 (AQP5), wherein numbering of amino acid positions is relative to the reference amino acid sequence of SEQ ID NO:9.

70. The method of aspect 67 or 68, wherein the peptide comprising the pathogenic T cell epitope comprises or consists of a sequence selected from the group consisting of SEQ ID NOS: 1-7.

71. The method of any one of aspects 67-70, wherein the MHC II is a human leukocyte antigen-DR 17 (HLA-DR17) isotype.

72. The method of aspect 71, wherein the HLA-DR17 isotype is a DRB1*0301 allele.

73. The method of any one of aspects 67-72, wherein the artificial antigen presenting cell is a MHC II I-A^{b} peptide tetramer.

74. The method of any one of aspects 67-73, wherein the natural antigen-presenting cell is a dendritic cell.

75. The method of any one of aspects 67-74, wherein the autoimmune aquaporinopathy is neuromyelitis optica spectrum disorder or Sjögren's syndrome.

It will be apparent to one of ordinary skill in the art that various changes and modifications can be made without departing from the spirit or scope of the invention.

### Experimental

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

### T Cell Deletional Tolerance Restricts AQP4 But Not MOG CNS Autoimmunity

### Introduction

Here, we examined how both central and peripheral mechanisms may contribute to T cell tolerance to AQP4 in WT mice. By examining peptide binding to purified MHC II molecules biochemically, we observed that the two pathogenic AQP4 T cell determinants bind I-A^{b} with higher affinity than nonpathogenic AQP4 T cell epitopes or the immunodominant encephalitogenic MOG p35-55. Single-cell T cell receptor sequencing (scTCR-Seq) was performed to examine AQP4-specific TCR α/β usage in combination with use of novel MHC II (I-A^{b}): AQP4 peptide tetramers to identify TCR-bearing cells that recognized the pathogenic AQP4 T cell epitopes. T cells from AQP4 i-mice selected an AQP4-specific TCR repertoire that was distinct from WT mice and exhibited clonal expansion. Collectively, these findings suggested that thymic negative selection may contribute to AQP4-specific T cell tolerance. Medullary thymic epithelial cells (mTECs), a cell type capable of expressing tissue-specific antigens (TSAs), including AQP4, are thought to direct negative selection by presenting those antigens in association with MHC molecules to CD4⁺ and CD8⁺ T cells, eliminating high affinity TCR-bearing self-antigen-specific T cells. Therefore, we created mice that were selectively deficient in mTEC expression of AQP4 and compared generation of AQP4-specific TCR-bearing (tetramer-positive, tet⁺) cells by AQP4 immunization in these mice, AQP4^{-/-} mice and WT mice. The frequency of peripheral tet⁺ T cells generated in AQP4^{-/-} mice was ten-fold higher than in WT mice. In comparison to WT mice, tet⁺ T cells were significantly elevated in mice selectively deficient in thymic AQP4 expression, but not to the level observed in AQP4^{-/-} mice. The autoimmune regulator, Aire and Fezf2, two transcription factors expressed by mTECs that direct negative T cell selection to many autoantigens, did not impact the frequency of tet⁺ T cells generated in comparison to WT mice. These findings suggest that thymic negative selection alone may not be the predominant mechanism of AQP4-specific T cell tolerance.

Expression of T cell-polarizing cytokines by pathogenic AQP4^{-/-} and nonpathogenic WT AQP4-specific T cells was similar, suggesting that resistance to CNS autoimmunity in WT mice was not due to an intrinsic inability to generate proinflammatory AQP4-reactive T cells or expansion of regulatory AQP4-specific T cells (18, 19). Thus, we questioned whether another process contributed to tolerance to AQP4. Donor AQP4-reactive Th17 cells were tested for their capability to induce clinical and histologic disease in recipient WT mice, in mice deficient in both T and B cells, and in mice lacking either T cells or B cells only. Recipient WT mice and mice containing T cells, but not B cells, developed clinical disease and CNS inflammation, but then recovered. In contrast, recipient mice deficient in both T and B cells, or mice lacking T cells only, developed persistent paralysis, suggesting that peripheral host T cells promoted recovery. Donor AQP4-specific T cells, like MOG-specific T cells, persisted in secondary lymphoid tissue and in the CNS of T cell-deficient mice. While donor AQP4-specific T cells were detected initially in recipient WT mice, there was a dramatic reduction during CNS disease and recovery, which was associated with an increased frequency of apoptotic cells among residual AQP4-reactive donor cells. By comparison, MOG-reactive T cells caused persistent paralysis in recipient WT mice and were detected in all compartments at each time evaluated. Our results indicate that peripheral T cell deletion has a prominent role in tolerance to AQP4, a member of the ubiquitous water channel family.

### Results

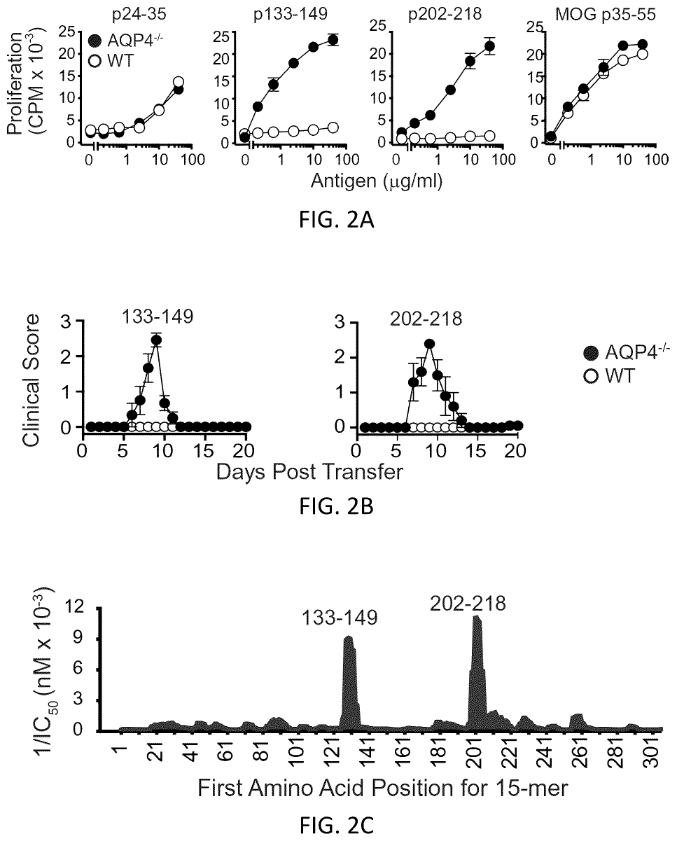

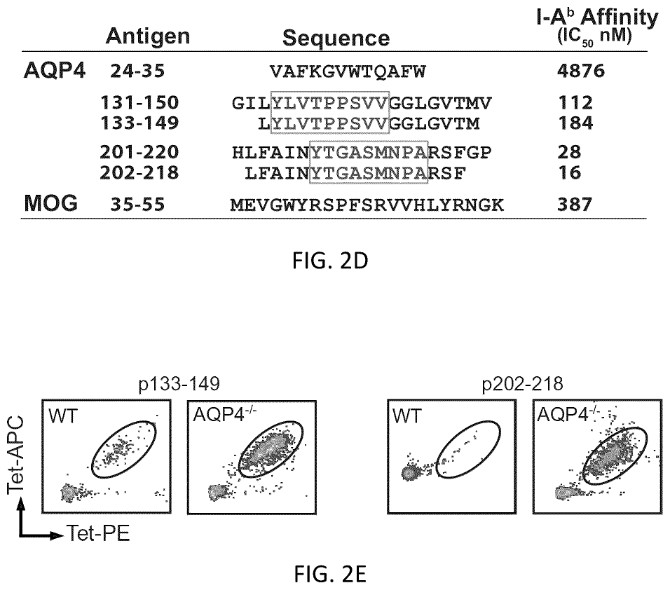

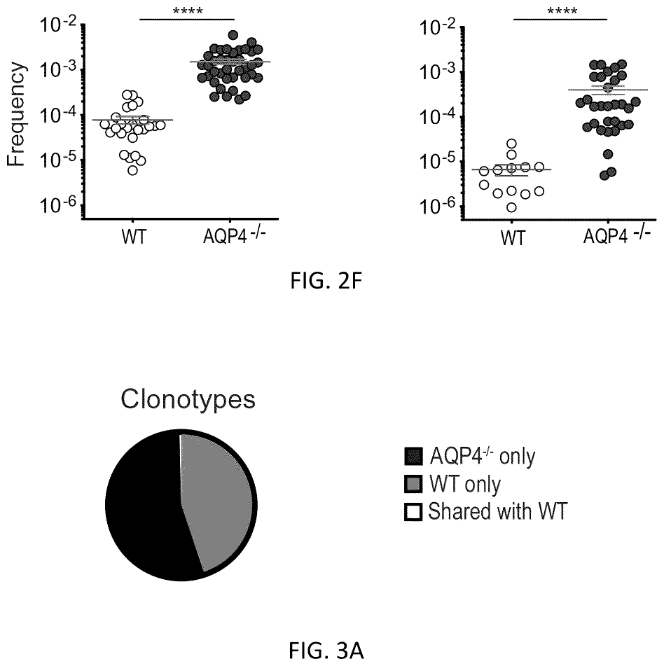

Pathogenic AQP4 T cell epitopes bind MHC II with high affinity. Pathogenic AQP4 T cell determinants located within amino acids 133-153 and 201-220 were discovered when studying immune responses in AQP4^{-/-} mice. Peptides p133-149 and p202-218, induce robust proliferative responses in AQP4^{-/-}, but not WT, mice (FIG. 2A) and Th17 cells specific for these AQP4 peptides are capable of inducing paralysis and CNS inflammation in WT mice (FIG. 2B). Discordance in proliferation between AQP4/and WT mice was not observed for nonpathogenic AQP4 T cell epitopes (e.g., AQP4 p24-35) (13) or encephalitogenic MOG p35-55.

Both AQP4 p133-149 and p202-218 are predicted to bind MHC II (I-A^{b}) with high affinity according to the Immune Epitope Database (IEDB) (FIG. 2C), an in silico method for predicting determinants within proteins that bind allele-specific MHC molecules for presentation to T cells (21). We have now examined AQP4 peptide affinity for I-A^{b} biochemically. I-A^{b} molecules that were purified by affinity chromatography were tested in a competitive binding assay between a radiolabeled high-affinity competitor peptide and unlabeled peptides (22). Indeed, peptides containing the two pathogenic AQP4 T cell determinants bound I-A^{b} with higher affinity than the nonpathogenic AQP4 p24-35 T cell epitope or MOG p35-55 (FIG. 2D). In fact, AQP4 p202-218 bound I-A^{b} with more than ten times greater affinity than MOG p35-55.

MHC II: peptide tetramers, I-A^{b}:AQP4 p133-149 and I-A^{b}:p202-218, were created in order to detect CD4⁺ T cells bearing TCRs specific for the two pathogenic AQP4 determinants. Analysis of primary lymph node CD4⁺ T cells from AQP4 peptide-immunized WT and AQP4-deficient mice identified a ten-fold higher frequency of proliferative tetramer-positive (tet⁺) T cells in AQP4-deficient mice (FIG. 2E). I-A^{b}:AQP4 p133-149 tet⁺-sorted T cells from p133-149-immunized AQP4^{-/-} mice transferred CNS autoimmunity to WT mice, although it was possible to expand tet pathogenic T cells (FIG. S1). Thus, tet+ status did not identify pathogenic T cells exclusively. Collectively, our observations that AQP4 p133-149 and p202-218 bound I-A^{b} avidly and that AQP4^{-/-}, but not WT, T cells for these determinants transferred CNS autoimmune disease to WT recipient mice suggested that generation of the pathogenic AQP4-specific T cell repertoire is prevented by negative selection.

### AQP4-Specific TCR Repertoire Selection in WT and AQP4^{-/-} Mice is Distinct.

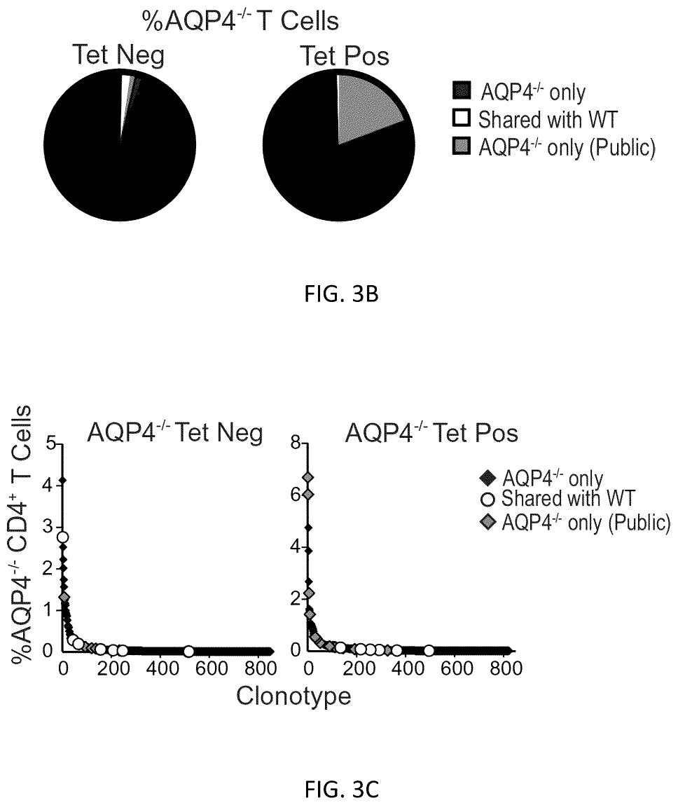

If loss of thymic negative selection permits expansion of pathogenic AQP4-specific T cells, one might predict that TCRs selected by AQP4-reactive T cells in AQP4-deficient and WT mice would differ. We evaluated this possibility by examining rearranged TCR α/β genes within individual AQP4-reactive T cells from AQP4 p133-149-primed AQP4-deficient and WT mice by scTCR-Seq using the 10× Genomics platform. Here, we focused on p133-149-specific T cells as those cells bound their corresponding tetramers more efficiently. Only a small fraction (0.3%) of AQP4-specific TCR α/β clonotypes examined were shared between WT and AQP4^{-/-} mice (FIG. 3A). AQP4-specific tet⁺ T cells from AQP4^{-/-} mice had minimal overlap between WT and AQP4-deficient T cells (FIG. 3B). The tet⁺ T cells from AQP4^{-/-} mice contained many expanded TCR clonotypes that were shared amongst multiple AQP4^{-/-} mice (“public” indicated in red) (FIG. 3C). In contrast, the AQP4-reactive tet T cells contained very few public TCR sequences, where overlap was primarily with WT T cells. Although this sampling represents a small proportion of the entire TCR repertoire, our results indicate that thymic negative selection is one mechanism that restricts development of pathogenic AQP4-reactive T cells in WT mice.

Absence of Thymic Negative Selection Alone does not Permit Expansion of AQP4-Reactive T Cells.

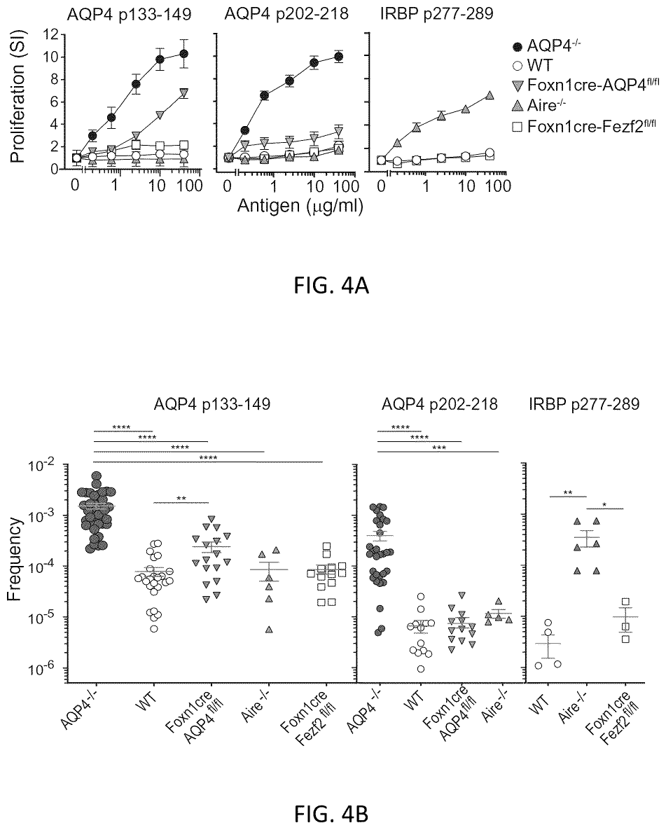

mTECs express peripheral TSAs for the purpose of presenting them and deleting self-antigen-reactive thymic T cells, a process known as negative selection (23). Aire and Fezf2 are two distinct transcription factors expressed by mTECs that control deletion of T cells reactive to a majority of TSAs (24, 25). Data suggest that mTEC AQP4 expression may be Aire-dependent (26) or Fezf2-dependent (25). To examine our hypothesis that thymic deletion constrains development of peripheral AQP4-reactive T cells, we created mice that were selectively deficient in mTEC AQP4 (Foxn1cre-AQP4/fl) expression (FIG. S2). We compared priming to the pathogenic T cell epitopes in those mice with WT and AQP4^{-/-} mice, as well as Aire^{-/-} mice and mice selectively deficient in mTEC Fezf2 (Foxn1cre-Fezf2^{fl/fl}) expression. AQP4 p133-149 induced robust proliferative T cell responses in AQP4^{-/-} mice and also induced proliferation in Foxn1cre-AQP4^{fl/fl} mice, but not to the same level as in AQP4^{-/-} mice (FIG. 4A). AQP4 p133-149 induced little or no proliferation in WT mice, Foxn1cre-Fezf2^{fl/fl} mice or Aire^{-/-} mice. Like AQP4 p133-149, p202-218 induced strong proliferation in AQP4^{-/-} mice, but only marginal responses in Foxn1cre-AQP4^{fl/fl} mice and no significant responses in WT or Aire^{-/-} mice.

The frequencies of AQP4 p133-149-specific and p202-218-specific CD4⁺ tet⁺ T cells in individual AQP4 p133-149- and p202-218-primed mice were examined with I-A^{b}: p133-149 and I-A^{b}: p202-218 tetramers, respectively (FIG. 4B). I-A^{b}: p133-149 tet⁺ T cells were significantly elevated in AQP4^{-/-} mice in comparison to WT, Foxn1cre-AQP4^{fl/fl}, Aire^{-/-} and Foxn1cre-Fezf2^{fl/fl} mice, results that were compatible with the proliferative T cell responses in these mice. Similarly, I-A^{b}: p202-218 tet⁺ T cells were elevated in AQP4^{-/-} mice in comparison to other strains. Interphotoreceptor retinoid-binding protein (IRBP), a self-antigen known to contain a T cell determinant, p277-289, which is Aire-dependent (27), was tested in parallel and, as anticipated, p277-289 immunization promoted expansion of CD4⁺ I-A^{b}:IRBP p277-289 tet⁺CD4⁺ T cells in Aire/mice. Thus, Fezf2 and Aire do not participate in negative selection to these AQP4-specific T cell epitopes in WT mice.

While frequencies of I-A^{b}: p133-149 tet⁺ T cells in Foxn1cre-AQP4^{fl/fl} mice were significantly lower than in AQP4^{-/-} mice, their frequencies were significantly elevated in comparison to AQP4 p133-149-specific T cells in WT mice, which was consistent with proliferative responses to this determinant in these mice (FIG. 4A). These two observations, along with TCR repertoire analysis, indicate that thymic negative selection does influence formation of the T cell repertoire to AQP4 p133-149. Of particular interest, Foxn1cre-AQP4^{fl/fl} mice were resistant to CNS autoimmunity by immunization with p133-149 (Table S1). Our results clearly demonstrate that central tolerance alone does not restrain development of pathogenic AQP4-reactive T cells in WT mice.

### Peripheral T Cell Deletion Restricts AQP4-Targeted CNS Autoimmune Disease.

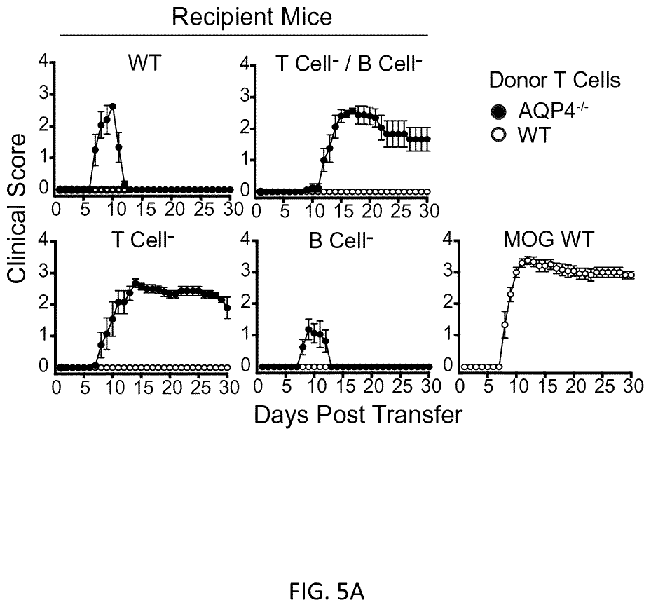

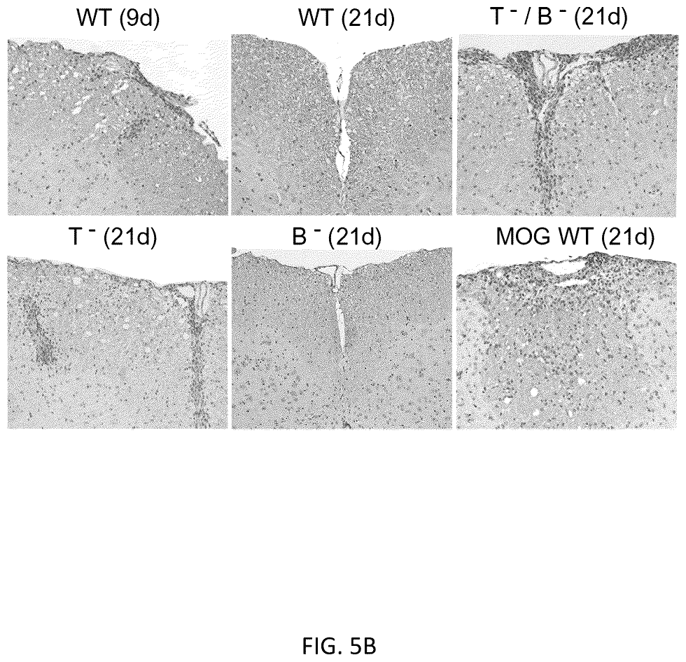

Previously, it was observed that pathogenic and nonpathogenic AQP4-specific T cells could not be distinguished based upon potential differences in production of proinflammatory or regulatory cytokines. Therefore, we questioned whether another mechanism of peripheral tolerance prevented AQP4-targeted disease induction in WT mice. Pathogenic AQP4-specific p133-149-specific Th17 cells from AQP4 mice were transferred to WT, T and B cell-deficient (RAG1^{-/-}), T cell-deficient (TCRa^{-/-}) or B cell-deficient (J_{H}T) mice. Recipient WT and J_{H}T mice developed paralysis beginning around day 7 after transfer, but then recovered completely 4-5 days later (FIG. 5A). CNS inflammation accompanied clinical disease (day 9) and was no longer evident after recovery (day 21) (FIG. 5B, Table S2). In contrast, recipient RAG1^{-/-} and TCRa^{-/-} mice developed persistent paralysis and CNS inflammation similar to WT mice that received donor MOG p35-55-specific Th17 cells.

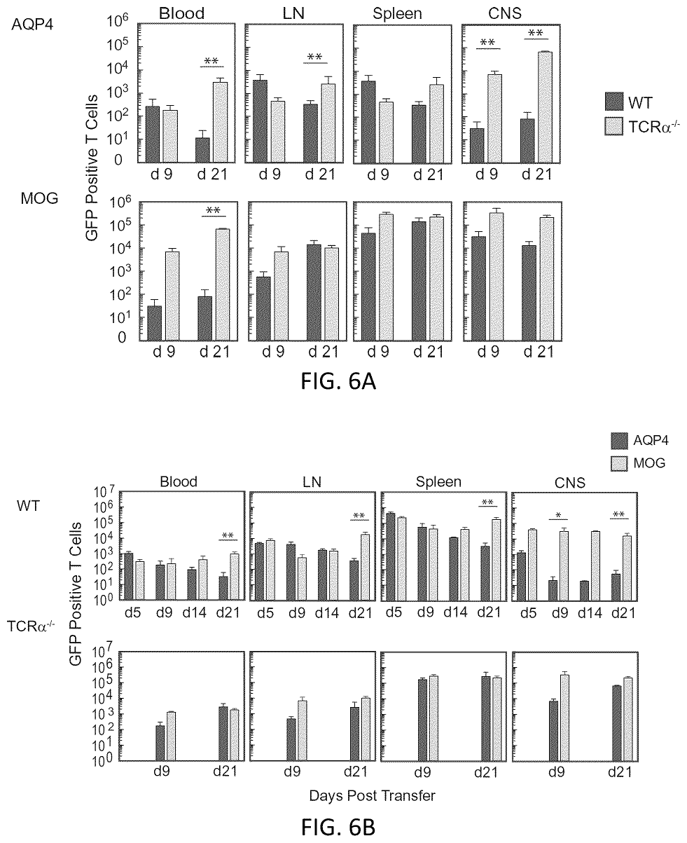

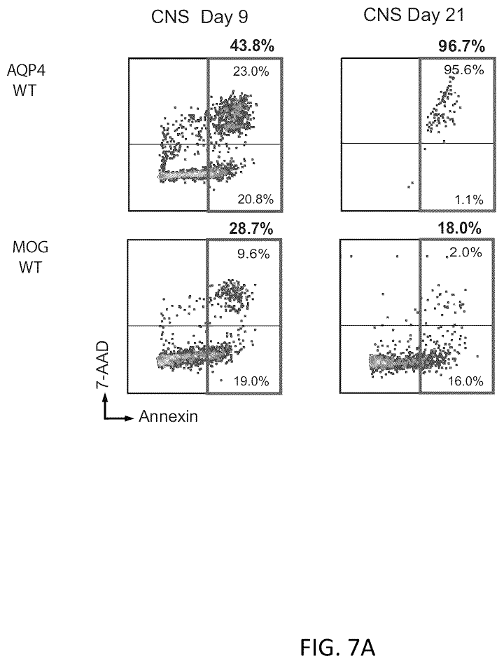

Our findings suggested that a peripheral T cell-dependent mechanism restricts AQP4-targeted CNS autoimmunity. In order to evaluate whether this process limited survival of AQP4-reactive T cells, we examined recovery of donor green fluorescent protein (GFP)⁺ pathogenic AQP4-specific T cells in TCRa^{-/-} and WT mice. There was a 2-3 log-fold reduction in recovery of donor T cells in WT recipient mice in comparison compared to TCRa^{-/-} mice (FIG. 6A). Donor GFP⁺ MOG-specific T cells were evaluated in parallel. Both donor AQP4-specific and MOG-specific T cells were detected in the CNS and secondary lymphoid tissues at multiple time-points in TCRa^{-/-} mice that developed persistent CNS autoimmune disease. Donor MOG-specific T cells were also detected in the CNS and secondary lymphoid tissues of WT recipient mice at all time points tested (FIG. 6B). In comparison, there was a 2-3 log-fold relative reduction in the number of donor AQP4-specific T cells in the CNS of WT mice during acute disease and recovery and a similar reduction of AQP4-specific donor T cells in secondary lymphoid tissues during recovery. Thus, in contrast to MOG-specific T cells, AQP4-specific T cells do not persist in WT hosts.

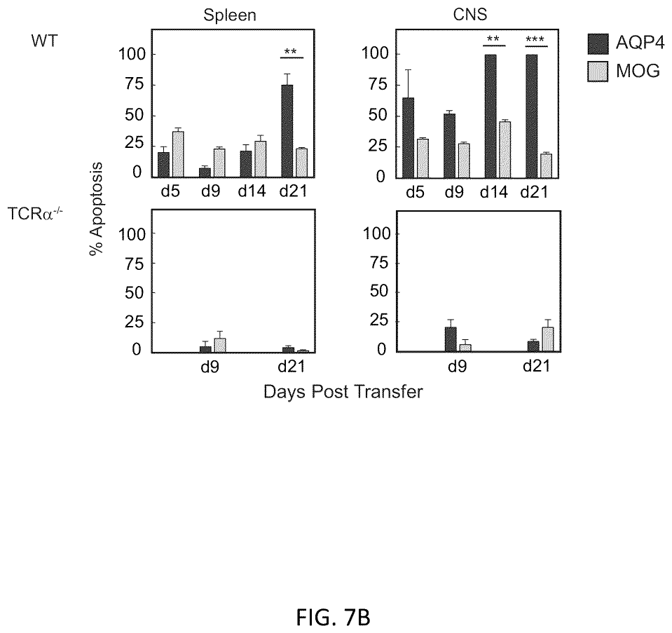

Annexin V staining was performed to determine if loss of donor AQP4-specific T cells in WT mice reflected increased apoptotic cell death in vivo (FIG. 7A). In contrast to donor MOG-specific T cells, there was a marked increased frequency of apoptotic AQP4-specific T cells in the CNS during disease and after recovery (FIG. 7B). The frequency of Annexin V⁺ donor MOG-specific and AQP4-specific T cells in recipient TCRa^{-/-} mice that developed persistent disease was low in both the spleen and the CNS and did not differ between groups. These findings indicate that AQP4-reactive T cells are more susceptible to peripheral deletion.

### Discussion

NMO and MOG antibody-associated disease (MOGAD) are two distinct CNS autoimmune inflammatory demyelinating diseases (28). Antibodies specific for APQ4 in NMO or MOG in MOGAD are IgG1, a T cell-dependent antibody isotype. The importance of humoral autoimmunity in NMO is underscored by the remarkable success of the complement inhibitor, eculizumab (29). However, only limited data support a pathogenic role for MOG-specific antibodies in MOGAD (30, 31). MOG was discovered as an autoantigen in EAE (32) more than a decade before anti-MOG antibodies were identified in acute disseminated encephalomyelitis (ADEM) (33), and nearly two decades before they were associated with optic neuritis and transverse myelitis (34, 35). Important lessons from MOG EAE can be applied to MOGAD (36-40). MOG EAE is a T cell-mediated disease, and while anti-MOG antibodies can exacerbate EAE and CNS demyelination, those antibodies are neither necessary, nor sufficient to cause EAE in the absence of MOG-specific T cells (30, 32, 39). Similarly, AQP4-specific antibodies are not pathogenic in the absence of CNS inflammation (8, 9). As for MOGAD, identifying mechanisms regulating T cell recognition of AQP4 in a model system may provide important conceptual advances for understanding the role of AQP4-specific T cells in NMO.

Recognition that WT mice were resistant to AQP4-induced CNS autoimmune disease and that pathogenic AQP4-specific T cells could be generated in AQP4^{-/-} but not in WT mice created a conundrum. Are AQP4-specific T cells restricted by central or peripheral tolerance? In this report, our findings that the AQP4-specific TCR repertoire selected in AQP4^{-/-} and WT mice were not the same and that there was a significant increase in AQP4 p133-149 tet⁺ T cells in thymic AQP4-deficient mice in comparison to WT mice, supported operation of a central tolerance mechanism. However, the numbers of p133-149 tet⁺ T cells in thymic AQP4-deficient mice were significantly lower than in AQP4^{-/-} mice, and thymic AQP4 deficiency did not permit significant restoration of AQP4 p202-218 tet⁺ T cells. Thus, a central mechanism alone did not shape the peripheral repertoire of AQP4-specific T cells. Demonstration that CNS autoimmunity in WT mice was self-limited and associated with reduced survival of AQP4-reactive T cells indicates that there is a dominant peripheral deletional mechanism distinct from MOG-targeted disease, which provides an additional layer of protection from AQP4 T cell autoimmunity.

Our observations that AQP4-targeted CNS autoimmunity was constrained by peripheral deletion of AQP4-reactive T cells and that this process was T cell-dependent are novel. However, peripheral apoptotic (e.g. FasL-induced) T cell deletion as may occur by repetitive stimulation with cognate antigen is well-recognized (41, 42). We have not yet identified which T cells may be responsible for apoptosis of pathogenic AQP4-reactive T cells. Recent studies have demonstrated that self-peptide-specific CD8⁺ T cells (43), including the Kir⁺CD8⁺ subset in humans and Ly49⁺CD8⁺ in mice, can suppress CD4⁺ antigen-specific T cell autoimmune responses (44, 45). Thus, in future studies we will investigate whether CD8⁺ T cells or another individual T cell subset may be responsible for deletion of pathogenic AQP4-reactive T cells.

IEDB, which was used as a resource for the initial characterization of the two pathogenic AQP4 T cell determinants, predicted that they bind MHC II I-A^{b} molecules with high affinity (18). Those predictions were validated biochemically in this report. It is important to recognize that the binary interaction of peptide with MHC II, a prerequisite for antigen presentation, does not necessarily translate to high avidity TCR engagement of MHC II: peptide complexes. We did not directly assess avidity of AQP4-specific TCRs for I-A^{b}:AQP4 peptide, although it is has been observed that high affinity self-antigen-reactive T cells are preferentially detected by MHC II: peptide tetramers (46). However, we have identified AQP4 p133-149-specific tet⁺ T cells that escaped negative selection in AQP4^{-/-} and Foxn1cre-AQP4^{fl/fl} mice, but not in WT mice. Thus, it should now be possible to determine whether AQP4-specific T cells subject to negative selection in the normal host express TCRs with high affinity for I-A^{b}:AQP4 p133-149 (47).

One potential limitation of this study is that we examined only a finite number of T cells by scTCR-Seq, yet the naive repertoire of potential TCR α/b clonotypes is immense (48). However, our goal was to focus on AQP4-specific T cells that were expanded by AQP4 priming in vivo. Further, our use of I-A^{b}:AQP4 tetramers in combination with scTCR-Seq permitted us to capture a large proportion of the polyclonal tet⁺ T cells. Although it is recognized that AQP4 is expressed most abundantly in the CNS, kidney, muscle and lung, it is not clear whether AQP4 is expressed in peripheral T cells. In previous work, we did not detect AQP4 protein or mRNA in WT murine peripheral T cells (49). However, one group reported that T cell AQP4 deficiency reduced TCR-mediated signaling, although they did not detect any change in thymocyte subsets (50). It should be recognized that it was the AQP4-specific T cells from AQP4^{-/-}, but not WT mice that induced CNS autoimmune disease. Donor T cells from AQP4 and WT mice did not differ in production of proinflammatory-polarizing and anti-inflammatory cytokines, or frequency of Foxp3⁺ regulatory T cells (18). Lastly, both donor AQP4-reactive and MOG-reactive T cells used in studies evaluating T cell survival in WT and TCRa^{-/-} mice were isolated from AQP4 mice. Thus, any potential change in TCR signaling would have been common to donor AQP4-specific and MOG-specific T cells.

AQP4 is expressed in multiple tissues, yet why is tissue damage in NMO considered primarily restricted to the CNS? Antibodies in NMO target membrane tetramers of AQP4 assembled into orthogonal arrays of particles (OAPs) that are expressed abundantly in astrocyte end-foot processes (51-53). OAPs provide an ideal substrate for binding of AQP4-specific antibodies, permitting cross-linking via C1q and activation of complement (54). NMO is a humoral OAP autoimmune disease. In contrast, AQP4-specific T cells recognize linear fragments of AQP4 in association with MHC molecules on APC, and not conformational determinants of AQP4 or OAPs. While AQP4-specific antibodies target astrocyte OAPs and account for the tissue specificity in NMO, T cells may be exposed to AQP4 in several tissues. We hypothesize that peripheral AQP4-specific T cell deletion provides a layer of protection against formation of AQP4-specific antibodies by B cells and the development of NMO. High affinity MHC-peptide binding may not only promote presentation, but also deletion. It may not be coincidence that the encephalitogenic AQP4 p202-218, which binds MHC II with highest affinity among peptides examined in this report, is remarkably homologous to amino acid sequences in aquaporins 1, 2, 5 and 6 (Table 1), aquaporins that are expressed ubiquitously. Thus, via “self-antigen mimicry” peripheral T cell deletion of AQP4-reactive T cells may provide tolerance to aquaporins, collectively. Conversely, a break in tolerance that permits T cell activation to an AQP4 epitope in one tissue may promote a proinflammatory response to AQP4, or a closely related sequence of another aquaporin, in a different organ. Both AQP4 and AQP5 are expressed in salivary glands (5). Thus, one can speculate that the Sjögren's syndrome-like salivary gland inflammation that is often associated with seropositive NMO (55-59) may be representative of an “autoimmune aquaporinopathy” (28), and sometimes even serve as a forme fruste forewarning the onset of NMO (60).

We have not created NMO in mice. However, we have provided a foundation to evaluate the regulation of AQP4-specific T cells in CNS autoimmunity, which may advance our understanding of aquaporin-specific T cells in NMO and in other autoimmune diseases.

### Materials and Methods

Mice. C57BL/6 (H-2^{b}), mice, B cell-deficient (J_{H}T), T cell-deficient (TCRa^{-/-}), T and B cell-deficient (RAG1^{-/-}) and Foxn1cre mice were purchased from the Jackson Laboratories (Bar Harbor, ME). C57BL/6 AQP4^{-/-} mice were provided by A. Verkman (UCSF), AQP4^{fl/fl} mice (61) were a gift from Ole Petter Ottersen, (University of Oslo), Fezf2^{fl/fl} mice (62) were from Nenad Sestan (Yale University), and Aire were provided by M. Anderson (UCSF). Foxn1cre-AQP4^{fl/fl} and Foxn1cre-Fezf2^{fl/fl} mice were generated by breeding female Foxn1cre mice with male AQP4^{fl/fl} or Fezf2^{fl/fl} mice respectively. Mice were housed under specific pathogen-free conditions at UCSF Laboratory Animal Research Center. All studies were approved by the UCSF Institutional Animal Care and Use Committee.

Antigens. AQP4 peptides p133-149, p202-218, p24-35, MOG p35-55, and IRBP p277-289 were synthesized by Genemed Synthesis.

I-A^{b} affinity Assay. Purification of MHC class II I-A^{b} molecules by affinity chromatography, and the performance of assays based on the inhibition of binding of a high affinity radiolabeled peptide to quantitatively measure peptide binding, were performed as described (22). Lower IC₅₀ values indicate higher binding affinity. Excellent binders have affinities less than 100 nM and good binders have affinities less than 1000 nM.

Proliferation assays. Mice were immunized subcutaneously (s.c.) with 100 μg of AQP4, MOG or IRBP peptide in CFA containing 400 μg M. tuberculosis H37Ra (Difco Laboratories). Lymph nodes were harvested on day 10-12 and cultured at 2×105/well with various concentrations of peptide in triplicate wells for 72 hours. 1 mci/well of ³H-thymidine was added 18 hours prior to cell harvest, and data are presented as counts per minute (CPM). Stimulation Index (SI) was calculated by dividing CPM in wells with Ag by CPM in wells with no antigen controls of each assay test group.

Flow cytometry. For analysis of tetramer binding, AQP4 p133-149, p202-218, IRBP p277-289 and hCLIP p87-101 IA^{b} tetramers conjugated to PE or APC were provided by the National Institute of Allergy and Infectious Diseases Tetramer Core Facility at Emory University. Lymphocytes were coincubated with 1 mg/ml tetramer conjugated to PE or APC for 2 hours at room temperature, washed and enriched using anti-PE magnetic microbeads (Miltenyi) over a magnetic column. Binding to hCLIP tetramer was used as a negative control. Dead cells were excluded using Live/Dead fixable Aqua Dead Cell Stain Kit (Invitrogen), lineage negative cells were excluded using antibodies to CD11b (M1/70), B220 (RA3-6B2) and CD8 (53-6.7) and positive cells were selected using antibodies to CD3 (145-2C11) and CD4 (RM4.5). 123count eBeads (Invitrogen) were used with unenriched and enriched samples for quantification of the frequency of tet⁺CD4⁺ T cells (total number of tet⁺CD4+ T cells divided by total CD4⁺ T cells per mouse). For figures showing tetramer staining, flow cytometry of the unenriched cells are shown. For analysis of GFP⁺ T cells, mice were perfused with PBS, and the CNS was dissected, minced, digested with collagenase and DNase I (Roche), and isolated over a Ficoll gradient as described previously (39). Live CD4⁺ cells were analyzed with a viability stain (Near-IR), and antibodies to CD11b, B220, CD8 and CD4. Apoptosis was analyzed separately using the same antibody staining, followed by Annexin V and 7-AAD staining in Annexin V binding buffer (BioLegend) per manufacturer's instructions. Cells were quantified using 123count eBeads.

Single cell TCR sequencing: AQP4^{-/-} and WT mice were immunized with AQP4 p133-149. T cells were harvested from draining lymph nodes and cultured with cognate antigen, receiving fresh antigen and irradiated splenocytes every 10 days for two stimulations. CD4⁺p133-149-specific AQP4^{-/-} T cells were sorted into tetramer positive or negative cells by flow cytometry. A single cell library of the T cell V(D)J regions targeting 10,000 cells from each group was constructed using the 10× Genomics Chromium Single Cell 5′ Library and Gel Bead Kit, and sequenced using the Illumina NovaSeq. Data shown represents two experiments.

Bioinformatic analysis. All datasets were analyzed using the Cell Ranger (v3.1.0) variable diversity joining (VDJ) function, which aligned reads to the GRCm38 Alts Ensembl reference (v3.1.0) using STAR (v2.5.1). Reads present in more than one sample that shared the same cell barcode and UMI were omitted using the SingleCellVDJdecontamination computational pipeline (https://github.com/UCSF-Wilson-Lab/SingleCellVDJdecontamination). TCR a and B chain contigs per cell, outputted from CellRanger, were further analyzed using custom bioinformatic programming scripts written in R and perl. For each cell, only one TCR α and one TCR β chain was kept by filtering for contigs assembled with the largest number of UMIs and raw reads. Cells were assigned to the same TCR clonotype if they share identical V genes, J genes and CDR3 amino acid sequences for both the TCR α and β chains. Cells with sequences that contained only a single chain or more than one TCR α and β chain were omitted, leaving 3000-6000 T cells per group. A clonotype was defined as clonally expanded if contained within two or more T cells (63, 64).

CNS autoimmune disease induction and analysis. For adoptive induction of CNS disease with primary lymphocytes, mice were immunized s.c. with 100 μg AQP4 or MOG peptides in CFA containing 400 μg M. tuberculosis H37Ra (Difco Laboratories). After 10-12 days, lymph node cells were cultured with 15 μg/ml antigen for 3 days with 20 ng/ml IL-23 and 10 ng/ml IL-6, under Tₕ17 polarizing conditions. 2×10⁷ cells were injected i.v. into naïve recipients, which then received 200 ng B. pertussis toxin (Ptx) (List Biological) i.p. on days 0 and 2. Clinical scores: 0=no disease, 1=tail tone loss, 2=impaired righting, 3=severe paraparesis or paraplegia, 4=quadraparesis, and 5=moribund or death.

For induction of CNS disease by I-A^{b}:AQP4 tet-sorted cells targeted cells, primary lymph node cells from AQP4 or WT mice were restimulated with 15 mg/ml antigen and irradiated splenocytes every 10 days. Percent tetramer binding was monitored by flow cytometry. In preparation for adoptive transfer, cells were washed and cultured with 1:10 T cell-to-irradiated splenocytes under polarizing conditions for 3 days. CD4⁺ cells were negatively sorted using magnetic beads (Miltenyi). 5×10⁶ were transferred to TCRa^{-/-} recipients. 200 ng Ptx was given on days 0 and 2.

Isolation of thymic epithelial cells (TECs): Thymi from 6-week-old AQP4^{-/-}, WT and Foxn1cre-AQP4^{fl/fl} mice were isolated, digested with 50 μg/ml liberase-TM (Roche) and 24 U/ml DNaseI (Sigma), stained with anti-CD45, anti-EpCAM, anti-Ly5.1, and anti-I-A^{b}, and separated by FACS cell sorting into mTEC^{hi}, mTEC^{lo} and cTEC cells. The mRNA was purified (Qiagen) and tested for AQP4 and GAPDH mRNA expression using ddPCR (Bio-Rad) per manufacturer's instructions.

Histopathology. Brain, spinal cord, optic nerve, kidney and muscle tissue samples were fixed in 10% neutral-buffered formalin, paraffin-embedded, sectioned and stained with H&E. Meningeal and parenchymal inflammatory lesions and areas of demyelination were assessed in a blinded manner as described (39).

Statistical analysis. Data are presented as mean±standard error of mean (SEM). Statistics for the frequency of T cell binding to tetramer and for the recovery of GFP⁺ T cells after adoptive transfer were calculated using the Mann Whitney non parametric T test. Statistics for the frequency of apoptotic cells in recovered tissues used a t-test with the Holm-Sidak correction for multiple comparisons. P values are designated as follows: *p<0.5, **p<0.01, ***p<0.001, ****p<0.0001.

TABLE 1
Homology of pathogenic AQP4 T cell epitopes with other aquaporins.

| | | | Identity‡ | | |
|---|---|---|---|---|---|
| Protein | Position* | Sequence*† | Sequence | Core | Organs§ |
| AQP4 | 133-149 | LYLVTPPSVVGGLGVT | | | CNS, kidney, salivary |
| | | (SEQ ID NO: 1) | | | gland, heart, GI tract, |
| | | | | | muscle, lung |
| AQP6 | 115-131 | LYGVTPGGIRETLGVN | 50% | 44% | Brain, kidney |
| | | (SEQ ID NO: 2) | | | |
| AQP4 | 202-218 | LFAINYTGASMNPARSF | | | |
| | | (SEQ ID NO: 3) | | | |
| AQP1 | 181-197 | LLAIDYTGCSINPARSF | 77% | 78% | CNS, kidney, heart, lung, |
| | | (SEQ ID NO: 4) | | | GI tract, ovary, testis, |
| | | | | | liver, muscle, ,spleen |
| AQP4 | 202-218 | LFAINYTGASMNPARSF | | | |
| | | (SEQ ID NO: 3) | | | |
| AQP2 | 173-189 | LLGIYFTGCSMNPARSL | 65% | 78% | Kidney, ear, ductus |
| | | (SEQ ID NO: 5) | | | deferens |
| AQP4 | 202-218 | LFAINYTGASMNPARSF | | | |
| | | (SEQ ID NO: 3) | | | |
| AQP5 | 174-190 | LVGIYFTGCSMNPARSF | 71% | 78% | Salivary gland, eye, |
| | | (SEQ ID NO: 6) | | | trachea, lung, GI tract, |
| | | | | | ovary, kidney |
| AQP4 | 202-218 | LFAINYTGASMNPARSF | | | |
| | | (SEQ ID NO: 3) | | | |
| AQP6 | 182-198 | LIGIYFTGCSMNPARSF | 71% | 78% | Brain, kidney |
| | | (SEQ ID NO: 7) | | | |

*Each mouse aquaporin was aligned individually to AQP4 using the Clustal Omega Multiple Sequence Alignment Tool (Clustal Omega < Multiple Sequence Alignment < EMBL-EBI) and homologous regions were identified.
†Identical residues are identified in bolded black and core binding regions are boxed.
‡The % homology for the sequence listed and the binding region is listed.
§(5)

### REFERENCES

1. P. Agre et al., Aquaporin water channels—from atomic structure to clinical medicine. J Physiol 542, 3-16 (2002).
2. A. Zekeridou, V. A. Lennon, Aquaporin-4 autoimmunity. Neurol Neuroimmunol Neuroinflamm 2, e110 (2015).
3. S. Nielsen et al., Specialized membrane domains for water transport in glial cells: high-resolution immunogold cytochemistry of aquaporin-4 in rat brain. J Neurosci 17, 171-180 (1997).
4. F. Magni et al., Proteomic knowledge of human aquaporins. Proteomics 6, 5637-5649 (2006).
5. A. K. Azad et al., Human Aquaporins: Functional Diversity and Potential Roles in Infectious and Non-infectious Diseases. Front Genet 12, 654865 (2021).
6. C. F. Lucchinetti et al., The pathology of an autoimmune astrocytopathy: lessons learned from neuromyelitis optica. Brain Pathol 24, 83-97 (2014).
7. R. I. Nurieva, Y. Chung, Understanding the development and function of T follicular helper cells. Cell Mol Immunol 7, 190-197 (2010).
8. J. L. Bennett et al., Intrathecal pathogenic anti-aquaporin-4 antibodies in early neuromyelitis optica. Ann Neurol 66, 617-629 (2009).
9. M. Bradl et al., Neuromyelitis optica: pathogenicity of patient immunoglobulin in vivo. Ann Neurol 66, 630-643 (2009).
10. D. G. Brum et al., HLA-DRB association in neuromyelitis optica is different from that observed in multiple sclerosis. Mult Scler 16, 21-29 (2010).
11. M. Varrin-Doyer et al., Aquaporin 4-specific T cells in neuromyelitis optica exhibit a Th17 bias and recognize Clostridium ABC transporter. Ann Neurol 72, 53-64 (2012).
12. A. Vaknin-Dembinsky et al., T-cell reactivity against AQP4 in neuromyelitis optica. Neurology 79, 945-946 (2012).
13. P. A. Nelson et al., Immunodominant T cell determinants of aquaporin-4, the autoantigen associated with neuromyelitis optica. PLOS One 5, e15050 (2010).
14. S. R. Kalluri et al., Functional characterization of aquaporin-4 specific T cells: towards a model for neuromyelitis optica. PLOS One 6, e16083 (2011).
15. B. Zeka et al., Highly encephalitogenic aquaporin 4-specific T cells and NMO-IgG jointly orchestrate lesion location and tissue damage in the CNS. Acta Neuropathol 130, 783-798 (2015).
16. M. Pohl et al., Pathogenic T cell responses against aquaporin 4. Acta Neuropathol 122, 21-34 (2011).
17. M. V. Jones, H. Huang, P. A. Calabresi, M. Levy, Pathogenic aquaporin-4 reactive T cells are sufficient to induce mouse model of neuromyelitis optica. Acta Neuropathol Commun 3, 28 (2015).
18. S. A. Sagan et al., Tolerance checkpoint bypass permits emergence of pathogenic T cells to neuromyelitis optica autoantigen aquaporin-4. Proc Natl Acad Sci USA 113, 14781-14786 (2016).
19. A. L. Vogel et al., Deletional tolerance prevents AQP4-directed autoimmunity in mice. Eur J Immunol 47, 458-469 (2017).
20. S. A. Sagan et al., Induction of Paralysis and Visual System Injury in Mice by T Cells Specific for Neuromyelitis Optica Autoantigen Aquaporin-4. J Vis Exp 10.3791/56185 (2017).
21. M. Mendes et al., IEDB-3D 2.0: Structural data analysis within the Immune Epitope Database. Protein Sci 10.1002/pro.4605, e4605 (2023).
22. J. Sidney et al., Measurement of MHC/peptide interactions by gel filtration or monoclonal antibody capture. Curr Protoc Immunol Chapter 18, Unit 18 13 (2013).
23. L. Klein, B. Kyewski, P. M. Allen, K. A. Hogquist, Positive and negative selection of the T cell repertoire: what thymocytes see (and don't see). Nat Rev Immunol 14, 377-391 (2014).
24. M. S. Anderson, M. A. Su, AIRE expands: new roles in immune tolerance and beyond. Nat Rev Immunol 16, 247-258 (2016).
25. H. Takaba et al., Fezf2 Orchestrates a Thymic Program of Self-Antigen Expression for Immune Tolerance. Cell 163, 975-987 (2015).
26. M. S. Anderson et al., Projection of an immunological self shadow within the thymus by the aire protein. Science 298, 1395-1401 (2002).
27. R. T. Taniguchi et al., Detection of an autoreactive T-cell population within the polyclonal repertoire that undergoes distinct autoimmune regulator (Aire)-mediated selection. Proc Natl Acad Sci USA 109, 7847-7852 (2012).
28. S. S. Zamvil, A. J. Slavin, Does MOG Ig-positive AQP4-seronegative opticospinal inflammatory disease justify a diagnosis of NMO spectrum disorder? Neurol Neuroimmunol Neuroinflamm 2, e62 (2015).
29. S. J. Pittock et al., Eculizumab in Aquaporin-4-Positive Neuromyelitis Optica Spectrum Disorder. N Engl J Med 381, 614-625 (2019).
30. M. Spadaro et al., Pathogenicity of human antibodies against myelin oligodendrocyte glycoprotein. Ann Neurol 84, 315-328 (2018).
31. J. J. Sabatino, Jr., A. K. Probstel, S. S. Zamvil, B cells in autoimmune and neurodegenerative central nervous system diseases. Nat Rev Neurosci 20, 728-745 (2019).
32. I. Mendel, N. Kerlero de Rosbo, A. Ben-Nun, A myelin oligodendrocyte glycoprotein peptide induces typical chronic experimental autoimmune encephalomyelitis in H-2b mice: fine specificity and T cell receptor V beta expression of encephalitogenic T cells. Eur J Immunol 25, 1951-1959 (1995).
33. K. C. O'Connor et al., Self-antigen tetramers discriminate between myelin autoantibodies to native or denatured protein. Nat Med 13, 211-217 (2007).
34. D. K. Sato et al., Distinction between MOG antibody-positive and AQP4 antibody-positive NMO spectrum disorders. Neurology 82, 474-481 (2014).
35. J. Kitley et al., Neuromyelitis optica spectrum disorders with aquaporin-4 and myelin-oligodendrocyte glycoprotein antibodies: a comparative study. JAMA Neurol 71, 276-283 (2014).
36. A. J. Slavin et al., Requirement for endocytic antigen processing and influence of invariant chain and H-2M deficiencies in CNS autoimmunity. J Clin Invest 108, 1133-1139 (2001).
37. E. Bettelli, D. Baeten, A. Jager, R. A. Sobel, V. K. Kuchroo, Myelin oligodendrocyte glycoprotein-specific T and B cells cooperate to induce a Devic-like disease in mice. J Clin Invest 116, 2393-2402 (2006).
38. G. Krishnamoorthy, H. Lassmann, H. Wekerle, A. Holz, Spontaneous opticospinal encephalomyelitis in a double-transgenic mouse model of autoimmune T cell/B cell cooperation. J Clin Invest 116, 2385-2392 (2006).
39. N. Molnarfi et al., MHC class II-dependent B cell APC function is required for induction of CNS autoimmunity independent of myelin-specific antibodies. J Exp Med 210, 2921-2937 (2013).
40. A. Shetty et al., Immunodominant T-cell epitopes of MOG reside in its transmembrane and cytoplasmic domains in EAE. Neurol Neuroimmunol Neuroinflamm 1, e22 (2014).
41. Y. Xing, K. A. Hogquist, T-cell tolerance: central and peripheral. Cold Spring Harb Perspect Biol 4 (2012).
42. M. A. ElTanbouly, R. J. Noelle, Rethinking peripheral T cell tolerance: checkpoints across a T cell's journey. Nat Rev Immunol 21, 257-267 (2021).
43. W. Yu et al., Clonal Deletion Prunes but Does Not Eliminate Self-Specific alphabeta CD8 (+) T Lymphocytes. Immunity 42, 929-941 (2015).
44. N. Saligrama et al., Opposing T cell responses in experimental autoimmune encephalomyelitis. Nature 572, 481-487 (2019).
45. J. Li et al., KIR(+) CD8(+) T cells suppress pathogenic T cells and are active in autoimmune diseases and COVID-19. Science 376, eabi9591 (2022).
46. J. J. Sabatino, Jr., J. Huang, C. Zhu, B. D. Evavold, High prevalence of low affinity peptide-MHC II tetramer-negative effectors during polyclonal CD4+ T cell responses. J Exp Med 208, 81-90 (2011).
47. W. A. Nyberg et al., An evolved AAV variant enables efficient genetic engineering of murine T cells. Cell 186, 446-460 e419 (2023).
48. M. K. Jenkins, H. H. Chu, J. B. Mclachlan, J. J. Moon, On the composition of the preimmune repertoire of T cells specific for Peptide-major histocompatibility complex ligands. Annu Rev Immunol 28, 275-294 (2010).
49. L. Li, H. Zhang, M. Varrin-Doyer, S. S. Zamvil, A. S. Verkman, Proinflammatory role of aquaporin-4 in autoimmune neuroinflammation. FASEB J 25, 1556-1566 (2011).
50. M. Nicosia et al., Water channel aquaporin 4 is required for T cell receptor mediated lymphocyte activation. J Leukoc Biol 10.1093/jleuko/qiad010 (2023).
51. J. E. Rash, T. Yasumura, C. S. Hudson, P. Agre, S. Nielsen, Direct immunogold labeling of aquaporin-4 in square arrays of astrocyte and ependymocyte plasma membranes in rat brain and spinal cord. Proc Natl Acad Sci USA 95, 11981-11986 (1998).
52. A. S. Verkman, J. Ratelade, A. Rossi, H. Zhang, L. Tradtrantip, Aquaporin-4: orthogonal array assembly, CNS functions, and role in neuromyelitis optica. Acta Pharmacol Sin 32, 702-710 (2011).
53. C. Palazzo et al., AQP4ex is crucial for the anchoring of AQP4 at the astrocyte end-feet and for neuromyelitis optica antibody binding. Acta Neuropathol Commun 7, 51 (2019).
54. J. Soltys et al., Membrane assembly of aquaporin-4 autoantibodies regulates classical complement activation in neuromyelitis optica. J Clin Invest 129, 2000-2013 (2019).
55. A. Javed et al., Minor salivary gland inflammation in Devic's disease and longitudinally extensive myelitis. Mult Scler 14, 809-814 (2008).
56. S. J. Pittock et al., Neuromyelitis optica and non organ-specific autoimmunity. Arch Neurol 65, 78-83 (2008).
57. J. M. Kahlenberg, Neuromyelitis optica spectrum disorder as an initial presentation of primary Sjogren's syndrome. Semin Arthritis Rheum 40, 343-348 (2011).
58. S. M. Kim et al., Sjogren's syndrome myelopathy: spinal cord involvement in Sjogren's syndrome might be a manifestation of neuromyelitis optica. Mult Scler 15, 1062-1068 (2009).
59. S. Jarius et al., Frequency and syndrome specificity of antibodies to aquaporin-4 in neurological patients with rheumatic disorders. Mult Scler 17, 1067-1073 (2011).
60. A. Jayarangaiah, R. Sehgal, N. Epperla, Sjogren's syndrome and neuromyelitis optica spectrum disorders (NMOSD)—a case report and review of literature. BMC Neurol 14, 200 (2014).
61. N. N. Haj-Yasein et al., Glial-conditional deletion of aquaporin-4 (Aqp4) reduces blood-brain water uptake and confers barrier function on perivascular astrocyte endfeet. Proc Natl Acad Sci USA 108, 17815-17820 (2011).
62. W. Han et al., TBR1 directly represses Fezf2 to control the laminar origin and development of the corticospinal tract. Proc Natl Acad Sci USA 108, 3041-3046 (2011).
63. J. A. Vander Heiden et al., pRESTO: a toolkit for processing high-throughput sequencing raw reads of lymphocyte receptor repertoires. Bioinformatics 30, 1930-1932 (2014).
64. N. Nouri, S. H. Kleinstein, A spectral clustering-based method for identifying clones from high-throughput B cell repertoire sequencing data. Bioinformatics 34, i341-i349 (2018).
65. M. Zimmermann, N. Meyer, Annexin V/7-AAD staining in keratinocytes. Methods Mol Biol 740, 57-63 (2011).
66. W. G. Telford, Multiparametric Analysis of Apoptosis by Flow Cytometry. Methods Mol Biol 1678, 167-202 (2018).

### Example 2

### Diagnosing Autoimmune Aquaporinopathy

Aquarporin-4 (AQP4) is the primary autoantigen in neuromyelitis optica (NMO). Up to 25% of NMO patients also develop Sjögren's sydrome. A diagnosis of NMO is established by antibodies to AQP4 as measured by cell-based assay (CBA). AQP5 may be a target for Sjögren's syndrome. NMO and Sjögren's syndrome are both associated with HLA-DR1*0301. Our research in mice and in humans (NMO patients) indicate that the pathogenic immune epitopes of AQP4 and AQP5 are closely related and that there could be cross-reactivity between the aquaporins. Thus, one may consider employing CBA for AQP5 diagnosis of Sjögren's syndrome, in particular in patients with NMO.

### Example 3

### Self-Antigen Mimicry Between the Two Autoantigens, AQP5 174-190 and AQP4 201-220

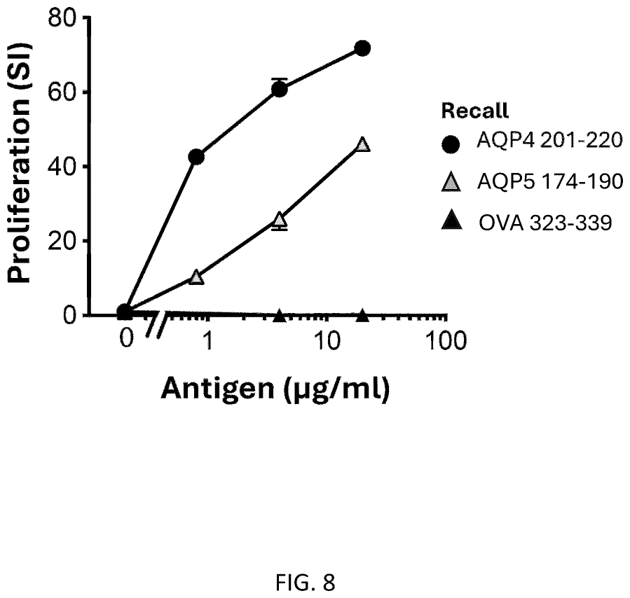

Aquaporin-4 (AQP4), expressed abundantly in the CNS, is the autoantigen in neuromyelitis optica (NMO) spectrum disorder (NMOSD). Patients with NMO frequently have other autoimmune conditions, including Sjögren's syndrome and systemic lupus erythematosus (SLE), which affect other tissues. AQP5, expressed in salivary and lacrimal glands, is an autoantigen in Sjögren's syndrome. Previously, we observed that the AQP4 T cell epitope (201-220) that causes CNS autoimmunity in mice binds major histocompatibility complex (MHC) class II molecules with exceptionally high affinity and is homologous to a sequence in AQP5, 174-190. Thus, we hypothesized that there may be T cell cross-reactivity (“self-antigen mimicry”) between the pathogenic epitope of AQP4 and AQP5, which could contribute to the development of both diseases in one individual. We have now tested for T cell cross-reactivity between AQP4 201-220 and AQP5 174-190. Our results shown in FIG. 8 demonstrate that AQP5 174-190 is stimulatory and that there is T cell cross-reactivity between AQP5 174-190 and AQP4 201-220, supporting self-antigen mimicry between these two autoantigens.

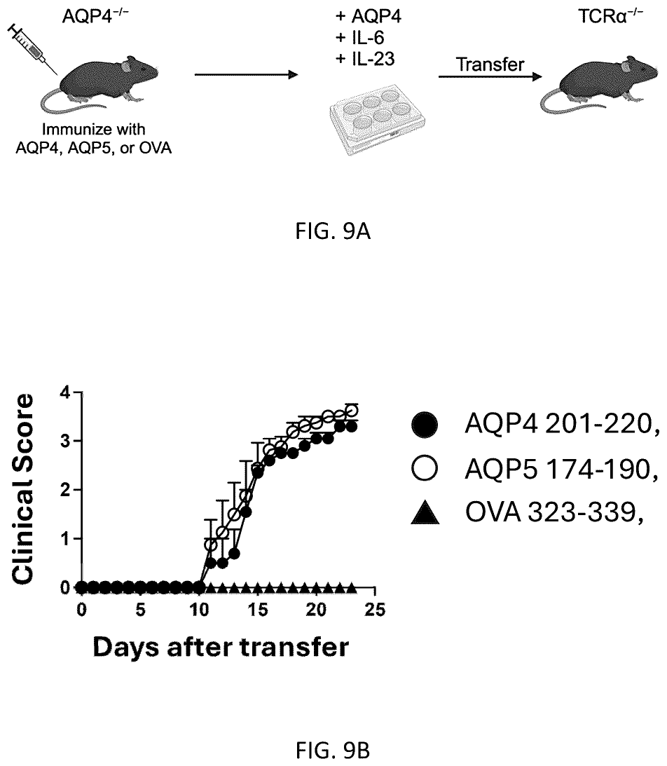

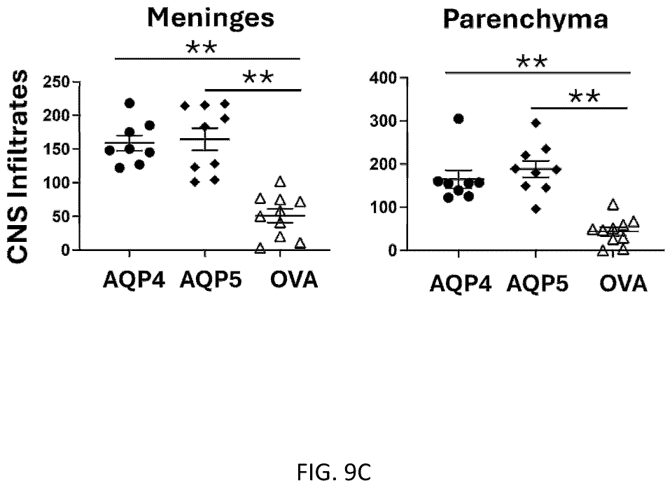

We have tested whether AQP5 174-190-primed T cells that cross-react with AQP4 201-220 can cause CNS autoimmune disease. As shown in FIGS. 9A-9C, T cells from AQP5 174-190-primed mice and expanded in response to AQP4 can induce clinical and histologic CNS autoimmune disease. In contrast, T cells from control antigen (OVA 323-336)-primed mice that were restimulated with AQP4 did not cause CNS autoimmune disease. Thus, our new findings demonstrate that individual T cells that target AQP5, an autoantigen in Sjögren's syndrome that is expressed in salivary and lacrimal glands, also recognize AQP4 and cause CNS autoimmune disease.

1. A method of detecting an autoreactive T cell associated with an autoimmune aquaporinopathy, the method comprising:
a) contacting a population of CD4⁺ T cells with a natural antigen-presenting cell or an artificial antigen-presenting cell, wherein the natural antigen-presenting cell or the artificial antigen-presenting cell comprises a class II major histocompatibility complex of an I-A^{b} haplotype (MHC II I-A^{b}) presenting a peptide comprising a pathogenic T cell epitope of an aquaporin; and
b) detecting binding of the autoreactive T cell, if present in the population of CD4+ T cells, to the pathogenic T cell epitope of the aquaporin or activation of the autoreactive T cell in response to the binding of the autoreactive T cell to the pathogenic T cell epitope of the aquaporin.

2. The method of claim 1, wherein the peptide comprising the pathogenic T cell epitope is selected from:
a peptide comprising or consisting of amino acids 133-149, 201-220, or 202-218 of aquaporin 4 (AQP4), wherein numbering of amino acid positions is relative to the reference amino acid sequence of SEQ ID NO:8;
a peptide comprising or consisting of amino acids 174-190 of aquaporin 5 (AQP5), wherein numbering of amino acid positions is relative to the reference amino acid sequence of SEQ ID NO:9; and
a peptide comprising or consisting of a sequence selected from the group consisting of SEQ ID NOS: 1-7.

3-4. (canceled)

5. The method of claim 1, wherein the MHC II is a human leukocyte antigen-DR 17 (HLA-DR17) isotype.

6. The method of claim 5, wherein the HLA-DR17 isotype is a DRB1*0301 allele.

7-8. (canceled)

9. The method of claim 1, wherein the autoimmune aquaporinopathy is neuromyelitis optica spectrum disorder or Sjögren's syndrome.

10. The method of claim 1, wherein said detecting activation of the autoreactive T cell comprises measuring proliferation, cytokine secretion, or expression of an activation marker.

11-12. (canceled)

13. The method of claim 1, wherein the autoreactive T cell is a type 1 helper T cell (Tₕ1) or a type 17 helper T cell (Tₕ17), wherein the Th1 or the Th17 is HLA-DR-restricted, wherein said detecting activation of the autoreactive T cell comprises detecting a Th1 immune response or a Th17 immune response, or a combination thereof.

14-15. (canceled)

16. The method of claim 13, wherein said detecting the Tₕ17 immune response comprises measuring Tₕ17 proliferation, cell surface expression of a transforming growth factor beta receptor, an interleukin 6 receptor, an alpha, interleukin 21 receptor, or an interleukin 23 receptor, or secretion of interleukin 17A (IL-17A), interleukin 17F (IL-17F), interleukin 17AF (IL-17AF), interleukin 21 (IL-21), or interleukin 22 (IL-22), or any combination thereof, and wherein said detecting the Th1 immune response comprises measuring Tₕ1 proliferation, cell surface expression of an interleukin 12 receptor beta 2, an interleukin 27 receptor alpha, an interferon gamma receptor 2, an interleukin 18 receptor, a C-C motif chemokine receptor 5 (CCR5), or a C-X-C motif chemokine receptor 3 (CXCR3), or secretion of interferon gamma (IFN-γ), tumor necrosis factor beta (TNF-β), interleukin 2 (IL-2), or interleukin 10 (IL-10), or any combination thereof.

17. (canceled)

18. A method of diagnosing and treating an autoimmune aquaporinopathy in a patient, the method comprising:
a) obtaining a biological sample comprising a population of CD4⁺ T cells from the patient;
b) contacting the population of CD4⁺ T cells with a natural antigen-presenting cell or an artificial antigen-presenting cell, wherein the natural antigen-presenting cell or the artificial antigen-presenting cell comprises a class II major histocompatibility complex of an I-A^{b} haplotype (MHC II I-A^{b}) presenting a peptide comprising a pathogenic T cell epitope of an aquaporin;
c) detecting binding of the autoreactive T cell, if present in the population of CD4+ T cells, to the pathogenic T cell epitope of the aquaporin or activation of the autoreactive T cell in response to the binding of the autoreactive T cell to the pathogenic T cell epitope of the aquaporin, wherein said detecting the binding to the pathogenic T cell epitope or the activation of the autoreactive T cell in response to the binding of the autoreactive T cell to the pathogenic T cell epitope of the aquaporin indicates that the patient has the autoimmune aquaporinopathy; and
d) treating the patient for the autoimmune aquaporinopathy if the patient is diagnosed as having the autoimmune aquaporinopathy based on said detecting the binding of the autoreactive T cell to the pathogenic T cell epitope of the aquaporin or the activation of the autoreactive T cell in response to the binding of the autoreactive T cell to the pathogenic T cell epitope of the aquaporin.

19. The method of claim 18, wherein the peptide comprising the pathogenic T cell epitope is selected from:
a peptide comprising or consisting of amino acids 133-149, 201-220, or 202-218 of aquaporin 4 (AQP4), wherein numbering of amino acid positions is relative to the reference amino acid sequence of SEQ ID NO:8;
a peptide comprising or consisting of amino acids 174-190 of aquaporin 5 (AQP5), wherein numbering of amino acid positions is relative to the reference amino acid sequence of SEQ ID NO:9; and
a peptide comprising or consisting of a sequence selected from the group consisting of SEQ ID NOS: 1-7.

20-21. (canceled)

22. The method of claim 18, wherein the MHC II is a human leukocyte antigen-DR 17 (HLA-DR17) isotype.

23. The method of claim 22, wherein the HLA-DR17 isotype is a DRB1*0301 allele.

24-25. (canceled)

26. The method of claim 18, wherein the autoimmune aquaporinopathy is neuromyelitis optica spectrum disorder or Sjögren's syndrome.

27. The method of claim 18, wherein said detecting activation of the autoreactive T cell comprises measuring proliferation, cytokine secretion, or expression of an activation marker.

28-29. (canceled)

30. The method of claim 18, wherein the autoreactive T cell is a type 1 helper T cell (Tₕ1) or a type 17 helper T cell (Tₕ17), wherein the Tₕ1 or the Tₕ17 is HLA-DR-restricted, wherein said detecting activation of the autoreactive T cell comprises detecting a Th1 immune response or a Th17 immune response, or a combination thereof.

31-32. (canceled)

33. The method of claim 30, wherein said detecting the Tₕ17 immune response comprises measuring Tₕ17 proliferation, cell surface expression of a transforming growth factor beta receptor, an interleukin 6 receptor, an alpha, interleukin 21 receptor, or an interleukin 23 receptor, or secretion of interleukin 17A (IL-17A), interleukin 17F (IL-17F), interleukin 17AF (IL-17AF), interleukin 21 (IL-21), or interleukin 22 (IL-22), or any combination thereof, and wherein said detecting the Th1 immune response comprises measuring Tₕ1 proliferation, cell surface expression of an interleukin 12 receptor beta 2, an interleukin 27 receptor alpha, an interferon gamma receptor 2, an interleukin 18 receptor, a C-C motif chemokine receptor 5 (CCR5), or a C-X-C motif chemokine receptor 3 (CXCR3), or secretion of interferon gamma (IFN-g), tumor necrosis factor beta (TNF-b), interleukin 2 (IL-2), or interleukin 10 (IL-10), or any combination thereof.

34. (canceled)

35. The method of claim 18, wherein the biological sample is obtained from blood, peripheral blood mononuclear cells (PBMCs), bone marrow, spleen, tonsils, or lymph nodes.

36. (canceled)

37. The method of claim 18, further comprising detecting an antibody that specifically binds to the aquaporin in the biological sample, wherein said detecting the antibody that specifically binds to the aquaporin in combination with said detecting the binding of the autoreactive T cell to the pathogenic T cell epitope of the aquaporin or the activation of the autoreactive T cell in response to the binding of the autoreactive T cell to the pathogenic T cell epitope of the aquaporin indicate that the patient has the autoimmune aquaporinopathy.

38. The method of claim 18, wherein said treating the patient for the autoimmune aquaporinopathy comprises performing plasma exchange, performing AQP4 or AQP5 immunoglobulin G (IgG) depletion or B cell depletion, performing stem cell transplantation, or administering a steroid, an immunosuppressive agent, an anti-mitotic agent, intravenous immunoglobulin (IVIG), a complement inhibitor, a calcineurin inhibitor, an inhibitor of guanosine nucleotide biosynthesis, an inhibitor of inosine monophosphate dehydrogenase, or an inhibitor of a folate-dependent enzyme, anti-IL-6 receptor therapy, or a combination thereof.

39-41. (canceled)

42. An artificial antigen-presenting cell comprising a class II major histocompatibility complex of an I-A^{b} haplotype (MHC II I-A^{b}) bound to a peptide comprising a pathogenic T cell epitope of an aquaporin, wherein the peptide comprising the pathogenic T cell epitope is selected from:
a peptide comprising or consisting of amino acids 133-149, 201-220, or 202-218 of aquaporin 4 (AQP4), wherein numbering of amino acid positions is relative to the reference amino acid sequence of SEQ ID NO:8;
a peptide comprising or consisting of amino acids 174-190 of aquaporin 5 (AQP5), wherein numbering of amino acid positions is relative to the reference amino acid sequence of SEQ ID NO:9; and
a peptide comprising or consisting of a sequence selected from the group consisting of SEQ ID NOS: 1-7.

43-45. (canceled)

46. The artificial antigen-presenting cell of claim 42, wherein the MHC II is a human leukocyte antigen-DR 17 (HLA-DR17) isotype.

47. The artificial antigen-presenting cell of claim 46, wherein the HLA-DR17 isotype is a DRB1*0301 allele.

48. The artificial antigen-presenting cell of claim 42, wherein the artificial antigen presenting cell is a MHC II I-A^{b} peptide tetramer.

49-56. (canceled)

57. A method of monitoring efficacy of a treatment of a patient for an autoimmune aquaporinopathy, the method comprising:
obtaining a first biological sample comprising a first population of CD4⁺ T cells from the patient before the patient undergoes the treatment and a second biological sample comprising a second population of CD4⁺ T cells from the patient after the patient undergoes the treatment;
contacting the first population of CD4⁺ T cells and the second population of CD4⁺ T cells with a natural antigen-presenting cell or an artificial antigen-presenting cell, wherein the natural antigen-presenting cell or the artificial antigen-presenting cell comprises a class II major histocompatibility complex of an I-A^{b} haplotype (MHC II I-A^{b}) presenting a peptide comprising a pathogenic T cell epitope of an aquaporin;
measuring amounts of autoreactive T cells bound to the pathogenic T cell epitope of the aquaporin in the first population of CD4⁺ T cells and the second population of CD4⁺ T cells; and
evaluating the efficacy of the treatment, wherein detection of increased amounts of the autoreactive T cells bound to the pathogenic T cell epitope of the aquaporin in the second population of CD4⁺ T cells compared to the first population of CD4⁺ T cells indicate that the patient is worsening or not responding to the treatment, and detection of decreased amounts of the autoreactive T cells bound to the pathogenic T cell epitope of the aquaporin in the second population of CD4⁺ T cells compared to the first population of CD4⁺ T cells indicate that the patient is improving; and
altering the treatment if the patient is worsening or not responding to the treatment.

58-65. (canceled)

66. The method of claim 57, wherein the autoimmune aquaporinopathy is neuromyelitis optica spectrum disorder or Sjögren's syndrome.

67-75. (canceled)


SEQUENCE LISTING (9 sequences)

SEQ ID NO: 1 - protein, 16 aa, synthetic construct
LYLVTPPSVVGGLGVT

SEQ ID NO: 2 - protein, 16 aa, synthetic construct
LYGVTPGGIRETLGVN

SEQ ID NO: 3 - protein, 17 aa, synthetic construct
LFAINYTGASMNPARSF

SEQ ID NO: 4 - protein, 17 aa, synthetic construct
LLAIDYTGCSINPARSF

SEQ ID NO: 5 - protein, 17 aa, synthetic construct
LLGIYFTGCSMNPARSL

SEQ ID NO: 6 - protein, 17 aa, synthetic construct
LVGIYFTGCSMNPARSF

SEQ ID NO: 7 - protein, 17 aa, synthetic construct
LIGIYFTGCSMNPARSF

SEQ ID NO: 8 - protein, 323 aa, Homo sapiens
MSDRPTARRWGKCGPLCTRENIMVAFKGVWTQAFWKAVTAEFLAMLIFVLLSLGSTINWGGTEKPLPVDMVLISLCFGLSIATMVQCFGHISGGHINPAVTVAMVCTRKISIAKSVFYIAAQCLGAIIGAGILYLVTPPSVVGGLGVTMVHGNLTAGHGLLVELIITFQLVFTIFASCDSKRTDVTGSIALAIGFSVAIGHLFAINYTGASMNPARSFGPAVIMGNWENHWIYWVGPIIGAVLAGGLYEYVFCPDVEFKRRFKEAFSKAAQQTKGSYMEVEDNRSQVETDDLILKPGVVHVIDVDRGEEKKGKDQSGEVLSSV

SEQ ID NO: 9 - protein, 265 aa, Homo sapiens
MKKEVCSVAFLKAVFAEFLATLIFVFFGLGSALKWPSALPTILQIALAFGLAIGTLAQALGPVSGGHINPAITLALLVGNQISLLRAFFYVAAQLVGAIAGAGILYGVAPLNARGNLAVNALNNNTTQGQAMVVELILTFQLALCIFASTDSRRTSPVGSPALSIGLSVTLGHLVGIYFTGCSMNPARSFGPAVVMNRFSPAHWVFWVGPIVGAVLAAILYFYLLFPNSLSLSERVAIIKGTYEPDEDWEEQREERKKTMELTTR